(19) United States
(12) Patent Application Publication
(10) Pub. No.: US 2026/0158269 A1
(43) Pub. Date: Jun. 11, 2026

(54) PROGRAMMING CLOSED-LOOP SPINAL CORD STIMULATION FOR SPASTICITY RELIEF

(71) Applicant: CLOSED LOOP MEDICAL PTY LTD, Macquarie Park, New South Wales (AU)

(72) Inventors: John Louis PARKER, Macquarie Park (AU); Gerrit Eduard GMEL, Macquarie Park (AU); Leonardo SILVESTRI, Macquarie Park (AU)

(21) Appl. No.: 19/126,924

(22) PCT Filed: Nov. 3, 2023
(86) PCT No.: PCT/AU2023/051112

§ 371 Date: May 2, 2025

(30) Foreign Application Priority Data
Nov. 4, 2022 (AU) 2022903306

(51) Int. Cl. A61N 1/36; A61N 1/372

(52) U.S. Cl. CPC A61N 1/36003; A61N 1/37241; A61N 1/3727

(57) ABSTRACT

Disclosed is a method for determining a recruitment target level (RTL) for programming spinal cord stimulation (SCS) to relieve spasticity in a muscle. The method includes controlling a stimulator device to apply probe stimuli of variable intensity to stimulate one or more afferent fibres of the muscle, and receiving values of intensity of neural responses measured via the stimulator device. The neural responses are evoked by respective probe stimuli in one or more efferent fibres of the muscle. The method further includes processing the neural response intensity values and the respective intensity values of the probe stimuli to determine a response growth curve indicating excitability of a stretch reflex of the muscle. An excitation response (ER) value is determined from the response growth curve, and the ER value is processed to generate the RTL as a target value for closed-loop control of the SCS.

The present application claims priority from Australian Provisional Patent Application No 2022903306 filed on 4 Nov. 2022, the contents of which are incorporated herein by reference in their entirety.

### TECHNICAL FIELD

The present invention relates to spinal cord stimulation for the relief of spasticity in a muscle, and in particular to the determination of a target level to enable closed-loop control of the stimulation.

### BACKGROUND OF THE INVENTION

There are a range of situations in which it is desirable to apply neural stimuli in order to alter neural function, a process known as neuromodulation. For example, neuromodulation is used to treat a variety of disorders including chronic neuropathic pain, Parkinson's disease, and migraine. A neuromodulation system applies an electrical pulse (stimulus) to neural tissue (fibres, or neurons) in order to generate a therapeutic effect. In general, the electrical stimulus generated by a neuromodulation system evokes a neural response known as an action potential in a neural fibre which then has either an inhibitory or excitatory effect. Inhibitory effects can be used to modulate an undesired process such as the transmission of pain, or excitatory effects may be used to cause a desired effect such as the contraction of a muscle.

In a number of neuromodulation systems, such as those configured to provide therapeutic pain relief, the electrical pulse is applied to the dorsal column (DC) of the spinal cord, a procedure referred to as spinal cord stimulation (SCS). Such a system typically comprises an implanted electrical pulse generator, and a power source such as a battery that may be transcutaneously rechargeable by wireless means, such as inductive transfer. An electrode array is connected to the pulse generator, and is implanted adjacent the target neural fibre(s) in the spinal cord, typically in the dorsal epidural space above the dorsal column. An electrical pulse of sufficient intensity applied to the target neural fibres by a stimulus electrode causes the depolarisation of neurons in the fibres, which in turn generates an action potential in the fibres. Action potentials propagate along the fibres in orthodromic (in afferent fibres this means towards the head, or rostral) and antidromic (in afferent fibres this means towards the cauda, or caudal) directions. Conventional neuromodulation systems stimulate fibres in this way, for example to inhibit the transmission of pain from a region of the body innervated by the target neural fibres (the dermatome) to the brain. To sustain the pain relief effects, stimuli are applied repeatedly, for example at a frequency in the range of 30 Hz-100 Hz.

In addition to applications for pain management, SCS also has utility in the treatment of muscle control disorders. Normal muscle tone in humans is maintained through a complex series of spinal reflexes and descending motor pathways. For example, one of the most important reflexes is through control of the spinal stretch reflex arc which is a closed neural loop that directly connects the muscle to the spinal cord via afferent (sensory) and back via efferent (motor) pathways without communication from the brain.

When a stretch reflex is activated, impulses are sent from the stretched muscle spindle via Ia afferent fibres to corresponding alpha-motoneuron fibres (α-MNs) of the muscle group. The α-MNs receive input from various pathways including one descending from the brain via the dorsal column, and without this descending input or with an insufficient descending input, a level of inhibition to the α-MNs may be reduced. This reduction in descending inhibition to the α-MNs in the spinal reflex arc may occur for certain muscles or muscle groups in response to an injury to the spinal cord or brain, either perinatal (e.g., cerebral palsy) or as a result of stroke. The stretch reflex arc then, in a neuroplastic response to this absence, becomes hyper-excitable for such muscle groups, keeping them in a permanent state of contraction known as spasticity. Spastic muscle groups in the limbs, in addition to being chronically painful, are of very little use for fine motor activities.

SCS has demonstrated an ability to provide relief of spasticity, and the pain associated with the condition, by stimulating nerve fibres (e.g. AB (A-beta) fibres) of the DC with the goal of compensating for the lack of inhibiting signals. For effective and comfortable SCS, it is necessary to maintain stimulus intensity above a threshold, such as to achieve “recruitment” of the DC nerve fibres. In almost all neuromodulation applications, response from a single class of fibre is desired, but the stimulus waveforms employed can evoke action potentials in other classes of fibres which cause unwanted side effects. It is therefore desirable to apply stimuli with intensity at a target value that does not significantly exceed the recruitment threshold, in order to avoid uncomfortable or painful percepts (e.g., due to over-recruitment of Aβ fibres).

The task of maintaining appropriate neural recruitment is made more difficult by electrode migration (change in position over time) and/or postural changes of the implant recipient (patient), either of which can significantly alter the neural recruitment arising from a given stimulus, and therefore negatively impact the ability to recruit the appropriate DC fibres to achieve relief of spasticity. There is room in the epidural space for the electrode array to move, and such array movement from migration or posture change alters the electrode-to-fibre distance and thus the recruitment efficacy of a given stimulus. Moreover, the spinal cord itself can move within the cerebrospinal fluid (CSF) with respect to the dura. During postural changes, the amount of CSF and/or the distance between the spinal cord and the electrode can change significantly. This effect is so large that postural changes alone can cause a previously effective stimulus regime to become either ineffectual or painful.

Many existing approaches to the application of SCS for spasticity relief are open-loop techniques in that the stimulation parameters are held fixed during the attempted recruitment of the DC fibres. A consequence is that some open-loop SCS treatment regimes, such as for cerebral palsy, are limited to an hour a day since adherence of the patient becomes impractical for longer periods (i.e., due to the level of discomfort experienced).

Moreover, the efficacy of open loop treatment depends on the stimulation intensity remaining appropriate throughout the treatment period. However, due to the aforementioned propensity for electrode migration, postural changes and/or movement of the patient, the ability to achieve DC fibre recruitment may be diminished resulting in the SCS treatment becoming ineffective or even detrimental (i.e., in the case of overstimulation events). These factors significantly impact the ability of open-loop approaches to SCS to provide effective relief of spasticity.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

In this specification, a statement that an element may be “at least one of” a list of options is to be understood to mean that the element may be any one of the listed options, or may be any combination of two or more of the listed options.

### SUMMARY OF THE INVENTION

According to a first aspect of the present technology, there is provided a method for determining a recruitment target level (RTL) for programming spinal cord stimulation (SCS) to relieve spasticity in a muscle, the method including: controlling a stimulator device to apply probe stimuli of variable intensity to stimulate one or more afferent fibres of the muscle; receiving values of intensity of neural responses measured via the stimulator device, the neural responses being evoked by respective probe stimuli in one or more efferent fibres of the muscle; processing the neural response intensity values and the respective intensity values of the probe stimuli to determine a response growth curve indicating excitability of a stretch reflex of the muscle; determining an excitation response (ER) value from the response growth curve; and processing the ER value to generate the RTL as a target value for closed-loop control of the SCS.

In some embodiments, the method further comprises receiving values of intensity of the applied probe stimuli measured via the stimulator device.

In some embodiments, the neural responses correspond to H-waves evoked in the one or more efferent fibres.

In some embodiments, the neural responses are evoked compound action potentials (ECAPs) in the one or more efferent fibres.

In some embodiments, the neural responses are EMGs in the one or more efferent fibres.

In some embodiments, the ER value is determined from a maximum slope of the response growth curve.

In some embodiments, the ER value is determined from a threshold of the response growth curve.

In some embodiments, the ER value is determined from a maximal amplitude of the evoked H-wave in the response growth curve.

In some embodiments, the ER value is further determined from a maximal amplitude of an evoked M-wave in the one or more efferent fibres.

In some embodiments, the ER value is determined as a ratio of the maximal amplitudes of the evoked H-wave and the evoked M-wave.

In some embodiments, generating the RTL comprises mapping the determined ER value to a corresponding target ECAP value.

In some embodiments, the target ECAP value is extracted from a predetermined response table including one or more candidate ER values and corresponding validated target ECAP values.

In some embodiments, the target ECAP value is determined by applying a response model to the determined ER value.

In some embodiments, the target ECAP value is determined by applying the response model to an ER differential value representing a difference between the determined ER value and an expected ER value of the muscle without spasticity.

In some embodiments, the response model is a linear regression model such that a magnitude of the target ECAP value is linearly proportional to the ER value.

In some embodiments, the response model is a set of classification parameters, such that the target ECAP value is output from a pattern classifier operating on the response model and an input of the ER value.

According to a second aspect of the present technology, there is provided a method for performing spinal cord stimulation (SCS) to relieve spasticity of a muscle, the method including: applying a therapeutic stimulus to the spinal cord to stimulate one or more afferent fibres associated with the muscle; measuring, in response to the application of the therapeutic stimulus, an intensity of a therapeutic neural response evoked by the therapeutic stimulus in the one or more afferent fibres; and adjusting an intensity of a subsequent therapeutic stimulus based on a feedback signal representing a difference between the measured therapeutic neural response intensity and an RTL determined by any of the methods described herein.

In some embodiments, the stimulator device conducts the applying, measuring, and adjusting to perform SCS to relieve spasticity in response to being programmed with the determined RTL.

In some embodiments, the method further includes adjusting the RTL before applying a subsequent therapeutic stimulus.

In some embodiments, adjusting the RTL comprises repeating the controlling, receiving, processing and determining to determine an updated excitation response (ER) value.

In some embodiments, adjusting the RTL further comprises: comparing the determined ER value to one or more other ER values associated with the treatment to relieve the spasticity of the muscle; and adjusting the RTL based on the comparison.

According to a third aspect of the present technology, there is provided a system for determining a recruitment target level (RTL) for programming spinal cord stimulation (SCS) to relieve spasticity in a muscle, the system including: a stimulator device comprising an electrode array and a pulse generator, the stimulator device configured to: apply, via the pulse generator, probe stimuli to stimulate one or more afferent fibres of the muscle via one or more probe stimulus electrodes of the electrode array; and measure, via one or more probe measurement electrodes of the electrode array, values of intensity of neural responses evoked by respective probe stimuli in one or more efferent fibres of the muscle, and a processor configured to: control the stimulator device to apply the probe stimuli at variable intensity and measure corresponding values of neural response intensity; process the neural response intensity values and the respective intensity values of the probe stimuli to determine a response growth curve indicating excitability of a stretch reflex of the muscle; determine an excitation response (ER) value from the response growth curve; and process the ER value to generate the RTL as a target value for closed-loop control of the SCS.

In some embodiments, the probe stimulus electrodes are implanted adjacent to the afferent fibres on the dorsal side of the spinal cord.

In some embodiments, the probe stimulus electrodes are implanted adjacent to the afferent fibres at a dorsal root of the spinal cord.

In some embodiments, the probe stimulus electrodes are located adjacent to the afferent fibres at the muscle.

In some embodiments, the probe measurement electrodes are implanted adjacent to the efferent fibres on the ventral side of the spinal cord.

In some embodiments, the probe measurement electrodes are implanted adjacent to the efferent fibres at a ventral root of the spinal cord.

In some embodiments, the probe measurement electrodes are located adjacent to the efferent fibres at the muscle.

In some embodiments, additional probe measurement electrodes are implanted adjacent to the afferent fibres at a dorsal root of the spinal cord.

In some embodiments, the neural responses correspond to H-waves evoked in the one or more efferent fibres.

In some embodiments, the ER value is determined from a maximal amplitude of an evoked H-wave in the response growth curve.

In some embodiments, the RTL is generated by mapping the determined ER value to a corresponding target ECAP value.

In some embodiments, the processor is further configured to program the stimulator device with the generated RTL.

In some embodiments, the stimulator device is further configured to: apply, via the pulse generator, a therapeutic stimulus to the spinal cord to stimulate one or more afferent fibres associated with the muscle via one or more therapeutic stimulus electrodes of the electrode array as a treatment to relieve the spasticity of the muscle; measure, via one or more therapeutic measurement electrodes of the electrode array, an intensity of a therapeutic neural response evoked by the therapeutic stimulus in the one or more afferent fibres; and adjust an intensity of a subsequent therapeutic stimulus based on the measured therapeutic neural response intensity, wherein the adjustment is based on a feedback signal representing a difference between the measured therapeutic neural response intensity and the determined RTL.

In some embodiments, the stimulator device is further configured to adjust the RTL before applying the subsequent therapeutic stimulus.

According to a fourth aspect of the present technology, there is provided a method for assessing a treatment to relieve spasticity in a muscle, the method including: (i) controlling a stimulator device to apply probe stimuli of variable intensity to stimulate one or more afferent fibres of the muscle; (ii) receiving values of intensity of neural responses measured via the stimulator device, the neural responses being evoked by respective probe stimuli in one or more efferent fibres of the muscle; (iii) processing the neural response intensity values and the respective intensity values of the probe stimuli to determine a response growth curve indicating excitability of a stretch reflex of the muscle; (iv) determining an excitation response (ER) value from the response growth curve; (v) comparing the determined ER value to one or more other ER values associated with the treatment to relieve the spasticity of the muscle; and (vi) determining a relative degree of spasticity of the muscle based on the comparing.

In some embodiments, the one or more other ER values define an expected range of ER values for the muscle without spasticity.

In some embodiments, the method for assessing a treatment to relieve spasticity in a muscle further includes iteratively repeating steps (i) to (vi), wherein the other ER values are previously determined ER values at step (iv).

In some embodiments, the relative degree of spasticity determined at each iteration of step (vi) is monitored to assess the treatment over time.

In some embodiments, the neural responses correspond to H-waves evoked in the one or more efferent fibres.

In some embodiments, the ER is determined from a maximum slope of the response growth curve.

In some embodiments, the ER is determined from a threshold of the response growth curve.

In some embodiments, the ER value is determined from a maximal amplitude of the evoked H-wave in the response growth curve.

According to a fifth aspect of the present technology, there is provided a device for assessing a treatment to relieve spasticity in a muscle, the device comprising: an electrode array; a pulse generator configured to apply probe stimuli to stimulate one or more afferent fibres of the muscle via one or more probe stimulus electrodes of the electrode array; and a control unit configured to: control the pulse generator to apply the probe stimuli at variable intensity; measure, via one or more probe measurement electrodes of the electrode array, values of intensity of neural responses evoked by respective probe stimuli in or more efferent fibres of the muscle; process the neural response intensity values and the respective intensity values of the probe stimuli to determine a response growth curve indicating excitability of a stretch reflex of the muscle; determine an excitation response (ER) value from the response growth curve; compare the determined ER value to one or more other ER values associated with the treatment to relieve the spasticity of the muscle; and determine a relative degree of spasticity of the muscle based on the comparing.

According to a sixth aspect of the present technology, there is provided a method for performing spinal cord stimulation (SCS) to relieve spasticity in a muscle, the method including: applying a therapeutic stimulus to the spinal cord to stimulate one or more afferent fibres associated with the muscle; measuring, in response to the application of the therapeutic stimulus, an intensity of a therapeutic neural response evoked by the therapeutic stimulus in the one or more afferent fibres; adjusting an intensity of a subsequent therapeutic stimulus based on a feedback signal representing a difference between the measured therapeutic neural response intensity and a recruitment target level (RTL); and adjusting the RTL before applying a subsequent therapeutic stimulus.

In some embodiments, adjusting the RTL comprises: determining an excitation response (ER) value for the muscle; comparing the determined ER value to one or more other ER values associated with the muscle; and adjusting the RTL based on the comparison.

In some embodiments, determining the ER value for the muscle comprises: controlling a stimulator device to apply probe stimuli of variable intensity to stimulate one or more afferent fibres of the muscle; receiving values of intensity of neural responses measured via the stimulator device, the neural responses being evoked by respective probe stimuli in one or more efferent fibres of the muscle; processing the neural response intensity values and the respective intensity values of the probe stimuli to determine a response growth curve indicating excitability of a stretch reflex of the muscle; and determining the ER value from the response growth curve.

According to a seventh aspect of the present technology, there is provided a device for performing spinal cord stimulation (SCS) to relieve spasticity in a muscle, the device comprising: an electrode array; a pulse generator configured to apply therapeutic stimuli to stimulate one or more afferent fibres associated with the muscle via one or more therapeutic stimulus electrodes of the electrode array; and a control unit configured to: control the pulse generator to apply a therapeutic stimulus; measure, via one or more therapeutic measurement electrodes of the electrode array, an intensity of a therapeutic neural response evoked by the therapeutic stimulus in the one or more afferent fibres; adjust an intensity of a subsequent therapeutic stimulus based on a feedback signal representing a difference between the measured therapeutic neural response intensity and a recruitment target level (RTL); and adjusting the RTL before applying the subsequent therapeutic stimulus.

According to an eighth aspect of the present technology, there is provided a method for performing spinal cord stimulation (SCS) to relieve spasticity in a muscle, the method including: (i) applying a therapeutic stimulus to the spinal cord to stimulate one or more afferent fibres associated with the muscle; (ii) determining an excitation response (ER) value for the muscle; (iii) comparing the determined ER value to one or more other ER values associated with the muscle; and (iv) adjusting an intensity of a subsequent therapeutic stimulus based the comparison.

In some embodiments, the method for performing SCS to relieve spasticity in a muscle further includes iteratively repeating steps (i) to (iv).

In some embodiments, determining the ER value for the muscle comprises: controlling a stimulator device to apply probe stimuli of variable intensity to stimulate one or more afferent fibres of the muscle; receiving values of intensity of neural responses measured via the stimulator device, the neural responses being evoked by respective probe stimuli in one or more efferent fibres of the muscle; processing the neural response intensity values and the respective intensity values of the probe stimuli to determine a response growth curve indicating excitability of a stretch reflex of the muscle; and determining the ER value from the response growth curve.

According to an ninth aspect of the present technology, there is provided a device for performing spinal cord stimulation (SCS) to relieve spasticity in a muscle, the device comprising: an electrode array; a pulse generator configured to apply therapeutic stimuli to stimulate one or more afferent fibres associated with the muscle via one or more therapeutic stimulus electrodes of the electrode array; and a control unit configured to: control the pulse generator to apply a therapeutic stimulus; determine an excitation response (ER) value for the muscle; compare the determined ER value to one or more other ER values associated with the muscle; and adjust an intensity of a subsequent therapeutic stimulus based the comparison.

References herein to estimation, determination, comparison and the like are to be understood as referring to an automated process carried out on data by a processor operating to execute a predefined procedure suitable to effect the described estimation, determination and/or comparison step(s). The technology disclosed herein may be implemented in hardware (e.g., using digital signal processors, application specific integrated circuits (ASICs) or field programmable gate arrays (FPGAs)), or in software (e.g., using instructions tangibly stored on non-transitory computer-readable media for causing a data processing system to perform the steps described herein), or in a combination of hardware and software. The disclosed technology can also be embodied as computer-readable code on a computer-readable medium. The computer-readable medium can include any data storage device that can store data which can thereafter be read by a computer system. Examples of the computer-readable medium include read-only memory (“ROM”), random-access memory (“RAM”), magnetic tape, optical data storage devices, flash storage devices, or any other suitable storage devices. The computer-readable medium can also be distributed over network-coupled computer systems so that the computer-readable code is stored and/or executed in a distributed fashion.

### BRIEF DESCRIPTION OF THE DRAWINGS

One or more implementations of the invention will now be described with reference to the accompanying drawings, in which:

FIG. 1 is a schematic illustrating an implanted spinal cord stimulator, according to one implementation of the present technology;

FIG. 2 is a block diagram of the stimulator of FIG. 1;

FIG. 3a is flow diagram of a method for relieving spasticity in a muscle of a patient based on closed-loop spinal cord stimulation (SCS), according to one implementation of the present technology;

FIG. 3b is an illustration of an idealised activation plot for one posture of a patient undergoing neural stimulation;

FIG. 4 is a flow diagram of a method for determining a recruitment target level (RTL) value for the method of FIG. 3a;

FIG. 5 is an illustration of the typical form of the spinal reflex arc in an individual;

FIG. 6 is an illustration of a cross-section of the spinal cord showing efferent pathways and afferent pathways;

FIG. 7a is an illustration of H- and M-waves evoked for the soleus muscle from posterior tibial nerve stimulation at the knee;

FIG. 7b is an illustration of example response growth curves of the H-wave and the M-wave for a peripheral muscular stimulus;

FIG. 8 is a flow diagram of a method for determining an excitation response (ER) value for determining a recruitment target level (RTL) value, according to one implementation of the present technology;

FIG. 9 is a flow diagram of a method for using a response model to determine the RTL value from an excitation response (ER) value, according to one implementation of the present technology;

FIG. 10a is a flow diagram of a method of performing closed-loop SCS therapy via a neuromodulation device for the treatment of muscle spasticity, according to one implementation of the present technology;

FIG. 10b is a block diagram of a neuromodulation system configured to perform the method of FIG. 10a;

FIG. 11 is a block diagram of a method of assessing the efficacy of a treatment to relieve spasticity, according to one implementation of the present technology; and

FIG. 12 is a flow diagram of a method of performing closed-loop SCS therapy via a neuromodulation device for the treatment of muscle spasticity, according to one implementation of the present technology.

### DETAILED DESCRIPTION OF THE PRESENT TECHNOLOGY

As an alternative to the open-loop SCS therapy for spasticity relief described above, performing SCS with closed-loop control enables the adjustment of the stimulation parameters to maintain a predetermined level of neural recruitment. Implementing closed-loop control has demonstrated the ability to address some of the drawbacks of open-loop SCS in the context of therapeutic pain management. Closed-loop control of an applied stimulus (i.e., a stimulus signal) is dependent on the ability to accurately measure the intensity of a neural response evoked by the stimulus (i.e., as a neural response signal). The neural response signal is measurable in terms of the action potentials generated by the depolarisation of a large number of fibres by the stimulus to form an evoked compound action potential (ECAP). Accordingly, an ECAP is the sum of responses from a large number of single fibre action potentials. The ECAP generated from the depolarisation of a group of similar fibres may be measured at a measurement electrode as a positive peak potential, then a negative peak, followed by a second positive peak. This morphology is caused by the region of activation passing the measurement electrode as the action potentials propagate along the individual fibres.

Approaches for obtaining a neural response measurement are described by the present applicant in International Patent Publication No. WO2012/155183, the content of which is incorporated herein by reference. In the context of relieving spasticity, the closed-loop control of SCS has been clinically shown to enable a much higher level of DC activation (nearly 10 times the average activation), and as a result has the potential to provide significantly greater descending inhibition thereby improving the potential to treat muscle spasticity (see Parker [1]). Significantly, in theory, a closed-loop SCS system will enable real-time instantaneous control of the level of DC recruitment, even during hyper-reflexive motion.

Despite the theoretical benefits, there are difficulties in programming a neuromodulation system to perform closed-loop SCS for spasticity relief. Specifically, there are no discernible trends or consensus on the stimulation parameters to use and no objective means to determine if the stimulation applied by the SCS system is reaching a level that is desired or targeted. Conventional closed-loop approaches for pain management use recruitment and discomfort thresholds to control the stimulus, where these thresholds can be empirically determined based on feedback from patients. However, many patients suffering from muscle spasticity are unable to provide feedback in a sufficiently accurate or reliable manner, thereby preventing the determination of an appropriate recruitment target level for SCS treatment of their spasticity. Furthermore, it is likely that the desired closed-loop target value for therapeutic benefit to spasticity will depend on both the muscle of interest and the condition of the individual (i.e., the degree to which their ability to provide descending inhibitions to the fibres is compromised). It is desired to ameliorate these drawbacks, or one or more other deficiencies of the previous approaches, or to at least provide a useful alternative.

### Overview

Disclosed herein are methods and systems for determining a target neural response value, referred to herein as the “recruitment target level”, enabling closed-loop control of a corresponding stimulus applied to the spinal cord to relieve spasticity in a muscle, or muscle group, without causing weakness or hypotonia in the muscle or muscle group. A stimulus of varying intensity level (e.g., increasing from zero, or a minimum value, to a maximum value) is applied to stimulate one or more afferent fibres of the muscle, such as Ia afferent nerve fibres, and intensities of a neural response evoked in one or more efferent fibres of the muscles, such as corresponding alpha-motoneuron (α-MN) fibres of the muscle, are measured. A response growth curve is generated from the neural response intensity values, and a degree of excitability of the stretch reflex is determined by the determination of an excitation response (ER) value from the curve. A recruitment target level (RTL) is obtained by processing the ER value, for example by translating or mapping the ER value into a value of a neural response parameter that is measurable by a closed-loop SCS system, such as for example an ECAP value.

The response growth curve is determined from amplitudes of the measured neural response signals that result from the Hoffman reflex (H-reflex) of the muscle of interest. The H-reflex is an artificial emulation of the stretch reflex that is triggered not by a stretching of the muscle spindle but by stimulation of the Ia afferent fibres over which the signal from the muscle spindle would travel. The H-reflex has been used to characterise the excitability of the stretch reflex (Palmieri [3]).

This growth curve therefore represents the stretch reflex of the muscle, and encapsulates the relationship between neural responses evoked in the Ia afferent fibres of the muscle and the resulting neural response of the α-MN fibres. That is, the response growth curve captures the characteristic of the excitability of the stretch reflex of the muscle as identified by a series of applied probe stimuli (to the Ia afferent fibres) and corresponding measured neural response values (from the α-MN fibres).

The ER value provides a metric to quantitatively evaluate stretch reflex excitability from the response growth curve of the α-MNs. In some embodiments, the ER is determined from the maximum response value of an indirectly evoked neural response of the α-MNs (i.e., the H-reflex response to the varying afferent fibre stimulus via the reflex arc) normalized by a corresponding maximal response value of a directly evoked neural response of the α-MNs (i.e. a muscle response to the varying afferent fibre stimulus). In the present disclosure, the indirect α-MN response is referred to as the H-wave and the direct α-MN response is referred to as the M-wave, though strictly speaking these terms are not generally used to refer to propagating action potentials. This is quantified by the ratio Hₘₐₓ/Mₘₐₓ of the H-wave peak amplitude to the peak amplitude of an M-wave in the muscle, as determined from the amplitude values of the respective response growth curves.

The H-reflex response, corresponding to evocation of the H-wave, may be achieved by stimulation of the dorsal roots of the dorsal column at an appropriate level to recruit an afferent pathway (e.g., the Ia afferent fibre) for the muscle.

In other embodiments, the ER value is derived from the minimal intensity of an afferent neural response (e.g. an ECAP) required to evoke the H-wave (the H-wave motor threshold). In such embodiments, there is no need to normalize such an ER value by a maximum M-wave response value. Other forms of the ER value may be derived from the H-wave response growth curve.

The determination of the ER value from a neural response growth curve, as performed by the proposed methods, devices and systems, advantageously provides a criterion for the quantitative measurement of the degree of excitability of the stretch reflex. Further, the ER value is insensitive to variability in measurements between individuals and between conditions on one individual, such as those resulting from variation in the electrode-cord distance. The ER values therefore consistently and quantitatively indicate the degree of spasticity of the muscle of a particular individual without requiring their subjective feedback.

In one application, the determined ER value, as determined from the neural response growth curve, is mapped to an RTL for programming a closed-loop SCS system to treat the spasticity in the muscle. In one embodiment, the RTL may be derived based on a predetermined relationship between the ER values and target ECAP values (e.g., as values stored in mapping table), as obtained from prior trial evaluations. Alternatively, a set of ER values and corresponding ECAP values may be obtained from healthy individuals (i.e., individuals with no spasticity in the muscle), enabling the training of a response model to produce the RTL (e.g., via regression or pattern classification) based on the ER value, or the difference between the ER value and an expected value in the healthy individuals. The closed-loop SCS system may be programmed with one or more response models that are trained offline, enabling generation of an RTL for an individual in real time, or substantially real time.

In other embodiments, the RTL may be derived by applying therapeutic stimuli of varying intensities, measuring the neural response intensities evoked by the therapeutic stimuli, and simultaneously and repeatedly determining ER values (i.e. to determine pairs of an ER value, as generated from a particular applied probe stimulus, and a corresponding measured neural response intensity). In response to the determined ER value reaching an acceptable level, based on all available data, the neural response intensity evoked by the present therapeutic stimulus is recorded as the RTL. The acceptable level of ER may be estimated based on clinical observation of the patient, e.g. as the ER value when the patient has the best outcome of the spasticity relief. Alternatively, the acceptable level of ER may be estimated from observations of healthy individuals.

A closed-loop SCS system may be programmed to use the target ECAP value (i.e., the RTL) to achieve and maintain an applied therapeutic stimulus aimed at treating the spasticity in the muscle. By repeatedly comparing the RTL to a measured ECAP, representing a neural response to the therapeutic stimulus, a corresponding feedback signal is generated by the SCS system to perform closed-loop control.

The determination of the RTL enables a practical implementation of closed-loop SCS for spasticity relief, and thereby addresses the disadvantages of the open-loop SCS approaches. Specifically, the RTL enables the intensity of the therapeutic stimulus applied by the system to be automatically adjusted such that treatment of spasticity may occur even in the presence of posture changes of a patient. Further, the treatment window available for a patient to receive SCS for relieving muscle spasticity may be significantly increased (e.g., from a single hour to a full 24 hours resulting in constant therapy throughout a day).

In another application, methods and systems are configured for assessing a therapeutic procedure or treatment for relieving muscle spasticity in an individual. The determination of the ER value from the neural response curve provides a means to quantitatively express a change in stretch reflex excitability of the muscle, as may occur progressively in response to the treatment. That is, by comparing an ER value presently derived from the patient with one or more other ER values associated with the treatment (e.g., nominal values expected from healthy individuals, or previous values obtained from the same patient), a relative degree of spasticity in the muscle can be inferred (e.g., from the difference in the ER values). The assessment may be performed for any therapeutic process applied with the goal of relieving spasticity, such as for example the administration of systemic agents to reduce hyper-excitability (benzodiazepines, baclofen), surgical procedures to ligate the dorsal afferent nerve (rhizotomy), and SCS either in open- or closed-loop modes of operation.

In some embodiments, the proposed SCS system is configured to generate an RTL specific to a spastic muscle of an individual (i.e., based on the determination of an α-MN response growth curve), and subsequently perform closed-loop SCS treatment with the determined RTL to relieve the spasticity of the muscle. In this sense, the system is advantageously capable of providing an integrated approach to spasticity treatment and assessment, involving programming a SCS system to: determine an ER value representing the current degree of muscle spasticity; determine if there is a significant difference of the determined ER value to one or more previously determined ER values; and if so, automatically adjust or reset the RTL based on the difference to improve the efficacy of closed-loop SCS treatment administered by the system thereafter.

In other embodiments, the ER value itself may be determined during therapy and compared with a desired ER value or range to provide a feedback signal to adjust the therapeutic stimulus intensity, thereby removing the need to measure the ECAP resulting from the therapeutic stimuli. In effect, the closed-loop SCS would operate by using the determined and desired ER values to regulate the applied therapeutic stimulus intensity, rather than using a measurement of the therapeutic neural response intensity.

### System for Relieving Spasticity

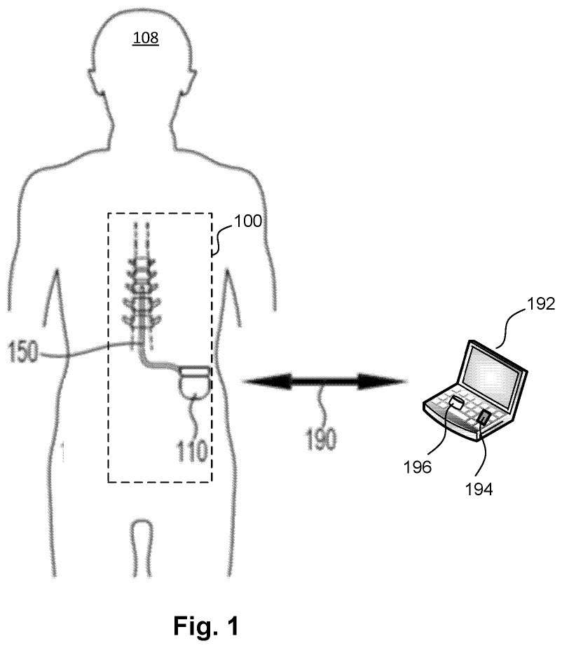

FIG. 1 schematically illustrates an embodiment a spinal cord stimulator 100, depicted as implanted in a patient 108, and a user device 192 that is external to the stimulator 100. The stimulator 100 and the user device 192 are collectively configured as part of a neuromodulation system for relieving spasticity via closed-loop spinal cord stimulation (CL-SCS).

Stimulator 100 comprises an electronics module (also referred to as a “control module”) 110 implanted at a suitable location. Stimulator 100 further comprises an electrode array 150, depicted as implanted within the epidural space, and connected to the module 110 by a suitable lead. The electrode array 150 may comprise one or more electrodes such as electrode pads on a paddle lead, circular (e.g., ring) electrodes surrounding the body of the lead, conformable electrodes, cuff electrodes, segmented electrodes, or any other type of electrodes capable of forming unipolar, bipolar or multipolar electrode configurations for stimulation and measurement. The electrodes may pierce or affix directly to the tissue itself.

Stimulator 100 operates as a neural modulation device that performs CL-SCS by: applying a stimulus to the spinal cord to stimulate one or more nerve fibres; and measuring a neural response signal that is evoked in response to the stimulus. For example, the neural response values may be measured as a compound action potential (CAP) that is evoked in response to the stimulus (referred to as an “ECAP”). An ECAP typically has a maximum amplitude in the range of microvolts, whereas an applied stimulus signal evoking the CAP is typically several volts.

Stimulator 100 is operable in a closed loop mode in which the intensity of the applied stimulus (e.g., the amplitude of a corresponding stimulus signal) is adjusted, or modulated, in response to a feedback signal. The feedback signal is determined from a difference between values of the measured neural response signal and a target value of the CL-SCS, such as the RTL in the embodiments discussed herein. This operation may also be referred to as closed loop neural stimulation (CLNS).

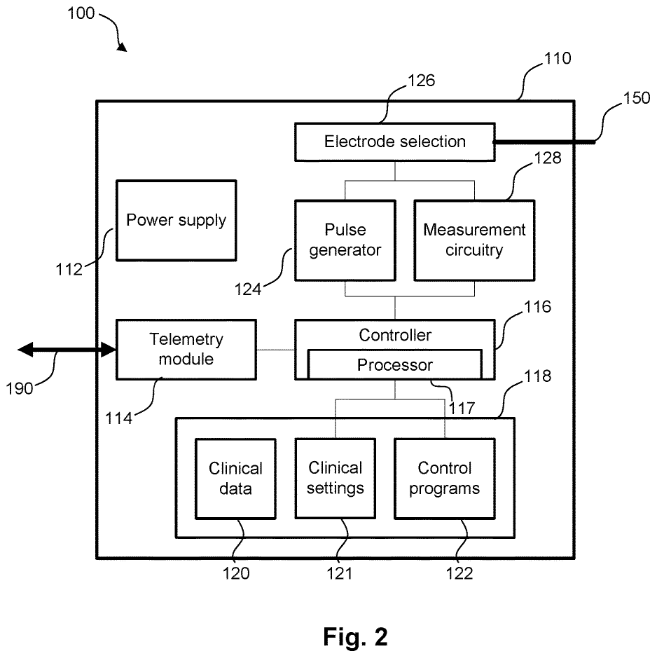

FIG. 2 is a block diagram of the stimulator 100. Electronics module 110 contains electronic components enabling the operation of stimulator 100. Electronics module 110 includes a battery 112 and a telemetry module 114. In implementations of the present technology, any suitable type of transcutaneous communications channel 190, such as infrared (IR), radiofrequency (RF), capacitive and inductive transfer, may be used by telemetry module 114 to transfer power and/or data to and from the electronics module 110 via communications channel 190.

Module controller 116 has an associated memory 118 storing one or more of clinical data 120, clinical settings 121, control programs 122, and the like. Controller 116 is configured by control programs 122, sometimes referred to as firmware, to control a pulse generator 124 to generate stimuli, such as in the form of electrical pulses, in accordance with the clinical settings 121. Controller 116 includes a processor 117 configured to execute one or more machine readable instructions of the control programs 122. The control programs 122 may include software programs written in a programming language such as C++ or Java, and configured, on execution, to instruct the processor 117 to perform the operations of method 300, or the associated sub-processes and methods.

Electrode selection module 126 switches the generated pulses to the selected electrode(s) of electrode array 150, for delivery of the pulses to the tissue surrounding the selected electrode(s). Measurement circuitry 128, which may comprise an amplifier and/or an analog-to-digital converter (ADC), is configured to process signals comprising neural responses sensed at measurement electrode(s) of the electrode array 150 as selected by electrode selection module 126.

The user device 192 is a computing device operable by a user, such as a clinician or the patient 108. In some embodiments, the user device 192 is a mobile computing device, such as a smart phone or tablet. In alternative embodiments, the user device 192 may be implemented as one or more full-scale computer devices, such as an Intel Architecture computer system configured as a desktop or laptop workstation.

In an exemplary configuration, user device 192 includes a processor 194 in communication with a memory system 196. The user device 192 further includes a networking system, one or more display interfaces, and one or more I/O device interfaces (not shown). The processor 194 may be any microprocessor which performs the execution of sequences of machine instructions, and may have architectures consisting of a single or multiple processing cores such as, for example, a system having a 32- or 64-bit Advanced RISC Machine (ARM) architecture (e.g., ARMvx). The processor 194 issues control signals to other device components via a system bus, and has direct access to at least some forms of the memory system 196.

Memory system 196 includes internal storage media for the electrical storage of machine instructions required to execute one or more software or firmware modules. For example, the internal storage media may include a combination of random access memory (RAM), non-volatile memory (such as ROM or EPROM), cache memory and registers, and high volume storage subsystems such as hard disk drives (HDDs), or solid state drives (SSDs). The modules stored in the memory system 196 include, but are not limited to, an operating system and one or more local application programs. For example, the local application programs may include, in some embodiments, programs for performing the operations of method 300, or the associated sub-processes and methods.

The user device 192 is connectable to one or more other computing devices and/or electronic modules via the networking system. A communications channel 190 connects the user device 192 to the module 110 of the stimulator 100. The communications channel 190 includes a wireless or wired transmission media enabling the exchange of data between the user device 192 and the module 110. The communications channel 190 may be implemented as a transcutaneous channel. Communications channel 190 may be active on a substantially continuous basis, at periodic intervals, at non-periodic intervals, or upon request from the device 192.

The stimulator 100 is programmable by the user device 192. In some embodiments, the user device 192 transmits data to the stimulator 100 to configure one or more of the control programs 122, that when executed by processor 117 control the operation of the stimulator 100. In one mode of operation, the implanted stimulator 100 operates to perform SCS on the patient 108 by: receiving control signals from the user device 192 instructing the application of a stimulus of a specified intensity; applying the stimulus via the operation of the pulse generator 124 and electrode array 150; and transmitting measurements of a neural response to the applied stimulus back to the device 192, via the communications channel 190.

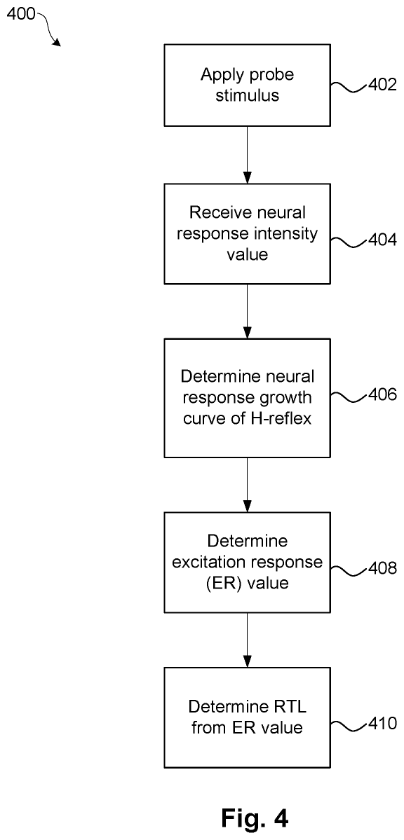

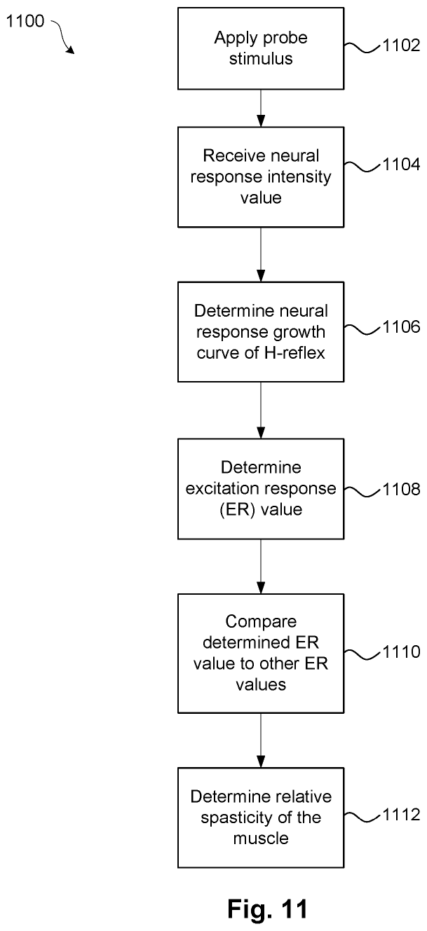

User device 192 may thus provide a clinical interface configured to program the implanted stimulator 100 and recover data stored on the implanted stimulator 100. This configuration is achieved by program instructions collectively referred to as the Clinical Programming Application (CPA) and stored in an instruction memory of the clinical interface. In such embodiments, the processor 194 of the user device 192 is configured to perform the methods to determine the RTL for use in closed-loop SCS, and to assess a therapy for treating spasticity (e.g., as depicted in FIGS. 4 and 11 respectively), by controlling the application of the stimulus by the stimulator 100 and receiving the corresponding neural response data.

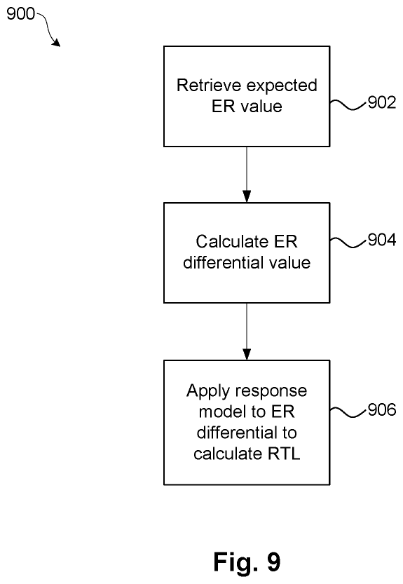

The user device 192 may further program the stimulator 100 to execute a method for performing CL-SCS to relieve spasticity in a muscle using a pre-determined RTL (as depicted in FIG. 9). For example, the RTL may be determined by the user device 192, based on neural response data obtained from the stimulator 100, and subsequently transmitted to the stimulator 100 via the communications channel 190.

In other embodiments, the processor 117 is configured to perform the methods described herein, including: actively determining the RTL for use in closed-loop SCS to relieve muscle spasticity; performing CL-SCS to relieve spasticity in a muscle based on the actively determined RTL; and assessing a therapy for treating spasticity based on actively determining one or more ER values. In such embodiments, the processor 117 of the stimulator 100 is configured to autonomously perform the processing of the neural response signal to determine a response growth curve, and derive the corresponding ER and RTL values. This mode of operation enables the configuration and execution of a closed-loop SCS treatment for muscle spasticity “on-line”, and in real time, by the stimulator 100. Further, the stimulator 100 may operate self-sufficiently to perform spasticity treatment and assessment functions (i.e., without further instruction or communication from the user device 192 once initially programmed).

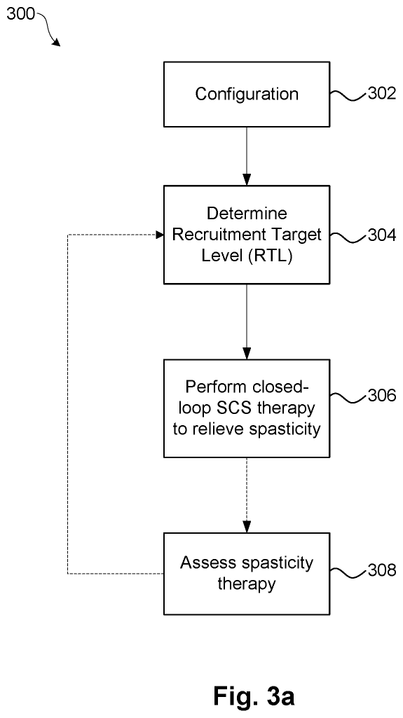

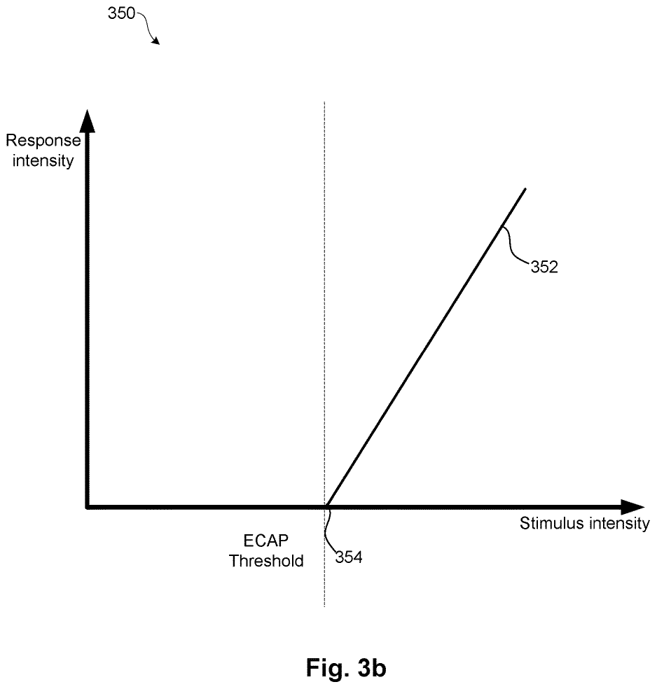

FIG. 3a illustrates a method 300 performed by a neuromodulation system for relieving spasticity in a muscle of a patient where the application of the closed-loop SCS (i.e., at step 306) is (optionally) integrated with an assessment of the therapy efficacy (i.e., at step 308) in a feedback process to influence the determination of a recruitment target level (RTL) for programming the SCS (i.e., at step 304).

### Configuration

At step 302, an initial configuration process is performed to configure stimulator 100 for operation to relieve spasticity for a muscle, or muscle group, of patient 108. The spinal cord stimulator 100 is implanted in patient 108, according to one implementation of the present technology. In one implementation, stimulator 100 is implanted in the patient's lower abdominal area or posterior superior gluteal region. In other implementations, the electronics module 110 is implanted in other locations, such as in a flank, or sub-clavicularly.

Electrode array 150 includes one or more electrodes (“probe stimulus electrodes”) that are collectively positioned to enable stimulation of at least one nerve fibre (e.g., Ia afferent fibres) of the spastic muscle (referred to herein as the “given muscle”), and one or more electrodes (“probe measurement electrodes”) that are collectively positioned to enable measurement of one or more corresponding neural responses to the stimulation, as described below. Electrode array 150 may also include one or more electrodes (“therapeutic electrodes”) that are collectively positioned to apply therapeutic stimuli to AB fibres in the dorsal column associated with the given muscle, and to measure one or more corresponding neural responses evoked by the therapeutic stimuli, as described below.

During operation, the stimulator 100 is configured to cause one or more electrodes (i.e., the probe or therapeutic stimulus electrodes) of the electrode array 150 to apply an electrical pulse to the target nerve fibres via activation of the pulse generator 124. The activation of the pulse generator 124 is controlled by controller 116, which is configurable to cause the generation of the applied pulse at a specified intensity. For example, the applied pulse may be a current pulse with the intensity corresponding to the pulse amplitude. The applied pulse causes the depolarisation of neurons, and generation of propagating action potentials thereby stimulating the nerve fibres. Delivery of an appropriate stimulus (i.e., of sufficiently high intensity) to the nerve evokes a neural response comprising an evoked compound action potential (ECAP). The stimulus electrodes are configurable to deliver stimuli periodically at any suitable frequency, for example 30 Hz, although other frequencies may be used including frequencies as high as the kHz range.

The neural response is detected by the measurement of an electrical field parameter signal by the measurement circuitry 128 components. For example, the measurement of the electrical field parameter signal may include the measurement of at least one of: an evoked neural compound action potential (ECAP); a non-evoked neural compound action potential (nECAP); a local field potential (LFP); a slow response; or another physiological signal (such as EMG, ECoG, and EKG). In the described embodiments, the stimulator 100 is configured to measure the intensity of neural responses in the form of ECAPs propagating along the target nerve fibres.

Probe stimulus electrodes are positioned in the dorsal epidural space above the DC for preferential recruitment of afferent fibres associated with the muscle. In some embodiments, the stimulation and measurement is localised to the DC, such that all electrodes of array 150 are positioned in the dorsal epidural space enabling any of the electrodes of the array 150 to be selected by the electrode selection module 126 to act as the probe measurement electrodes to measure the neural response resulting from the applied stimulus. In other embodiments, some probe stimulus electrodes may be positioned: (i) peripherally, near the spastic muscle or muscle group; or (ii) dorsoventrally, including at both the dorsal and the ventral sides of the spinal cord.

The measurement circuitry 128 components may be configured to perform differential measurement of the ECAP values. Differential ECAP measurements are less subject to common-mode noise on the surrounding tissue than single-ended ECAP measurements. The measured ECAP may be parametrised by any suitable parameter(s), including, for example, an amplitude of first and second positive peaks P1 and P2, an amplitude of a negative peak N1, or a peak-to-peak amplitude (as described in International Patent Publication No. WO2015/074121, the contents of which are incorporated herein by reference). Although the embodiments described herein relate to the measurement of an ECAP, the skilled addressee will appreciate that measurement of any other type of electrical field parameter indicating a neural response may be performed alternatively, or in addition.

The relationship between the stimulus intensity (e.g. an amplitude of an applied current pulse signal) and the intensity of the neural response evoked by the stimulus (e.g. an ECAP amplitude) is represented by an activation plot, or “growth curve”. FIG. 3b illustrates an exemplary activation plot 350 for one posture of the patient 108. The activation plot 350 shows a monotonic (e.g. linearly increasing) ECAP amplitude for stimulus intensity values above a threshold 354 referred to as the ECAP threshold. The ECAP threshold exists because of the binary nature of fibre recruitment; if the field strength is too low, no fibres will be recruited. However, once the field strength exceeds a threshold, fibres begin to be recruited, and their individual evoked action potentials are independent of the strength of the field. The ECAP threshold 354 therefore reflects the field strength at which significant numbers of fibres begin to be recruited, and the increase in response intensity with stimulus intensity above the ECAP threshold reflects increasing numbers of fibres being recruited. Below the ECAP threshold 354, the ECAP amplitude may be taken to be zero. Above the ECAP threshold 354, the activation plot 350 has a positive, approximately constant slope 352 indicating a linear relationship between stimulus intensity and the ECAP amplitude.

To relieve spasticity, it is desired to achieve and maintain the therapeutic response intensity at, or just above, a threshold value sufficient to cause restoration of inhibition (as described below). In the closed-loop mode of operation for performing a SCS therapy, the stimulator 100 adjusts the intensity of an applied therapeutic stimulus based on a measured response intensity parameter (i.e., a measured ECAP amplitude) and a target response intensity during the therapy. For example, the processor 117 may be configured to calculate an error between a target ECAP value and a measured ECAP amplitude, and adjust the applied therapeutic stimulus intensity to reduce the error as much as possible, such as by adding the scaled error to the current therapeutic stimulus intensity. The measured response intensity, and its deviation from the target response intensity, is used by the feedback loop to determine possible adjustments to the applied therapeutic stimulus intensity to maintain the measured response intensity at the target response intensity.

The stimulator 100 is configured to apply a stimulus to a target nerve fibre as a sequence of electrical pulses according to a predefined stimulation pattern. The stimulation pattern is characterised by multiple stimulus parameters including for example, an intensity value (i.e., pulse amplitude), pulse width, number of phases, order of phases, number of stimulus electrode poles (two for bipolar, three for tripolar etc.), and stimulus rate or frequency. At least one of the stimulus parameters, for example the stimulus intensity parameter, is controlled by the feedback loop.

In the configuration stage of step 302, the stimulator 100 is programmed with the set of stimulus parameters. For example, to determine the RTL a user may configure the stimulator 100 to deliver “probe” stimuli of over a range of incrementally increasing intensity values. The corresponding neural response intensity values may be used to form the response growth curve indicating stretch reflex excitability (as described below). The stimulus parameters may be loaded into the memory 118 of the stimulator 100 as the clinical settings 121 by a data exchange with the user device 192, as operated by the user (e.g., a clinician).

As part of configuration step 302, the user may program the control programs 122 of stimulator 100 and/or application programs of user device 192 with one or more parameters related to the spasticity condition of the patient 108. For example, stimulus parameters, including the set of stimulus intensities, for determining the RTL may be set based on the muscle or muscle group to be treated. In some embodiments, neural response measurements obtained from prior therapy performed on the patient 108, and/or the spastic muscle(s), may be used to directly set, or inform a selection of, the stimulus parameters.

### Determining Stretch Reflex Excitability

Following configuration (step 302 of the method 300), the stimulator 100 and user device 192 may be collectively operated to determine an RTL value to program closed-loop SCS for the treatment of muscle spasticity in the patient 108 (i.e., at step 304 of FIG. 3a). FIG. 4 illustrates a method 400 for determining the RTL value. In the described embodiments, the steps of method 400 are performed by processor 117 of the stimulator 100. Controller 116 is configured to operate the stimulator 100 according to a control program 122 configured at step 302 to determine the RTL for the given muscle of patient 108 (e.g., via a data exchange with the user device 192). In other embodiments, one or more of the steps of method 400 are performed by the processor 194 of the user device 192, as enabled by data exchanges with the stimulator 100.

At step 402, processor 117 is configured to control the stimulator 100 to apply a probe stimulus to stimulate one or more Ia afferent nerve fibres of the muscle.

Pulse generator 124 applies the probe stimulus to the Ia afferent nerve fibres via the probe stimulus electrodes of array 150, potentially evoking an ECAP in the Ia afferents and thereby also potentially evoking an H-wave in the α-MNs via the H-reflex. The probe stimulus electrodes may be located at the dorsal roots where the Ia afferent fibres enter the dorsal column. Alternatively, the probe stimulus electrodes may be located peripherally, at the given muscle itself. In this case, since the Ia afferents are bundled with the α-MNs at the periphery, stimulating the Ia afferents will also recruit the α-MNs directly, potentially evoking an M-wave in the α-MNs.

At step 404, the processor 117 receives a value of neural response intensity measured by measurement circuitry 128 in response to the applied probe stimulus. Measurement circuitry 128 measures a neural response intensity, for example an ECAP amplitude, of the H-wave evoked in the α-MNs by the applied probe stimulus (i.e., the current pulse of a given amplitude) via the probe measurement electrodes of the array 150. The probe measurement electrodes may be located at the ventral roots where the α-MNs exit the dorsal column, in order to sense the H-wave in the α-MNs. Alternatively, the probe measurement electrodes may be located peripherally, at the given muscle itself. In this case, the probe measurement electrodes may be transcutaneous or surface electrodes, configured to sense EMGs rather than ECAPs. EMGs are not propagating action potentials, but for the present disclosure they are included under the rubric of neural responses.

In embodiments in which the probe stimulus electrodes are located so as to evoke an M-wave as well as an H-wave, the measurement circuitry 128 may be configured to distinguish between the H-wave and the M-wave, for example based on the shorter latency of the M-wave. The measurement circuitry 128 may in such embodiments be configured to measure the intensity of the M-wave in addition to that of the H-wave, so that the intensities of both the H-wave and the M-wave are received by the processor 117 as response data.

In some embodiments, additional probe measurement electrodes may also be located, e.g. at the dorsal root, so as to measure the neural response evoked in the Ia afferents by the probe stimulus. In such embodiments, the intensities of the probe stimuli may be recorded in the probe stimulus data as the intensities of the neural responses (e.g., ECAP amplitudes) evoked in the Ia afferents by the probe stimuli, rather than as the amplitudes of the applied stimulus current pulses. Such a measure of stimulus intensity is robust to varying distance between the probe stimulus electrodes and the Ia afferent fibres and other variables that affect the stimulus intensity-response intensity relationship.

The steps 402 and 404 are repeated iteratively, for progressively increasing intensities of the probe stimulus. The controller 116 activates the pulse generator 124 to apply a series of probe stimuli, each at a particular intensity of a set of intensity values. The intensity values are specified by the control program 122 of the RTL determination routine (e.g., as defined by the user and uploaded to the stimulator 100 via device 192 at step 302). The application of the series of probe stimuli results in the generation of a corresponding series of the measured neural response intensity values (i.e., a neural response intensity value is generated in response to each applied probe stimulus of the particular intensity).

In the described embodiments, controller 116 is configured to represent the intensities of the i-th applied probe stimulus and the corresponding neural response intensity values as respective data values Sᵢ and Rᵢ for i=1 . . . . N. In the described embodiments, controller 116 is configured to store the probe stimulus data S and response data R in a data structure, such as an array, list, or table, in the memory of controller 116 such as to enable retrieval by the processor 117. In some embodiments, the probe stimulus and response data are transmitted by the telemetry module 114, and via channel 190, to the user device 192 for storage and/or processing.

The generation of the probe stimulus and response data is based on the spinal reflex arc, and particularly the responses of (Ia) afferent and (α-MN) efferent fibres of the muscle. The spinal stretch reflex arc (or monosynaptic stretch reflex) is a closed neural loop that directly connects the muscle to the spinal cord via the afferent and efferent pathways. The activation of the corresponding α-MNs controls the function of the muscle.

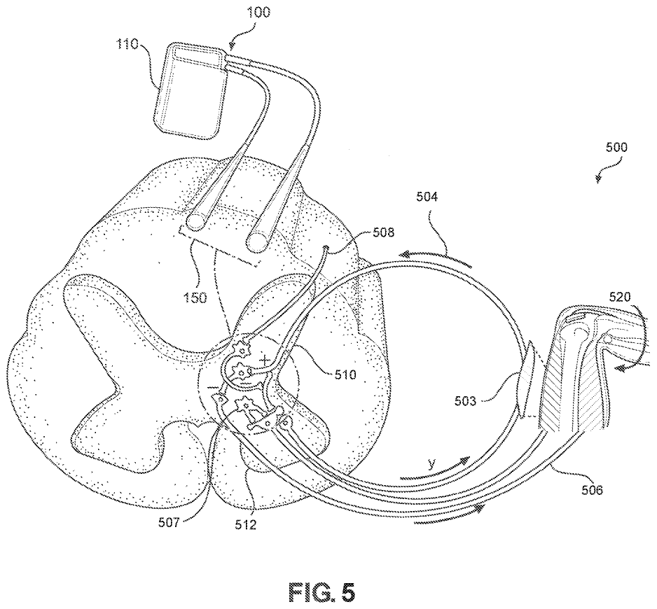

FIG. 5 illustrates the spinal reflex arc 500 in a healthy individual. The reflex begins when the muscle spindle 503 detects a change in muscle length corresponding to a stretching of the muscle 520. In response the Ia afferent fibres 504 are activated. The Ia afferent fibres transmit these sensory impulses to the dorsal horn of the spinal cord and excite the motor (muscle) efferents (α-MNs) 506 of the same muscle, thus causing the muscle to contract. At the same time, these afferents also inhibit α-MNs of the antagonist or opposing muscle through inhibitory interneurons 507, causing it to relax. Cutaneous afferents from skin mechanoreceptors 508 also enter the spinal cord at the dorsal root entry zone 510 and are known to also connect with the inhibitory interneurons 507.

The spinal reflex arc may become hyper-excitable as a result of insufficient control from the brain, i.e. a lack of descending inhibition to the α-MNs. SCS of the dorsal column to stimulate the cutaneous afferent fibres such as Aβ fibres has been shown to be effective at restoring a level of inhibition to the α-MNs. FIG. 5 shows the positioning of the electrodes of array 150 to stimulate the cutaneous afferent fibre(s) in the dorsal column.

In some described embodiments, stimulation and measurement of the (Ia) afferent and (α-MN) efferent fibres respectively is achieved by a dorsoventral configuration of the electrodes of array 150 involving the positioning of electrodes of array 150 near the ventral roots 512 as well as on the dorsal side of the spinal cord.

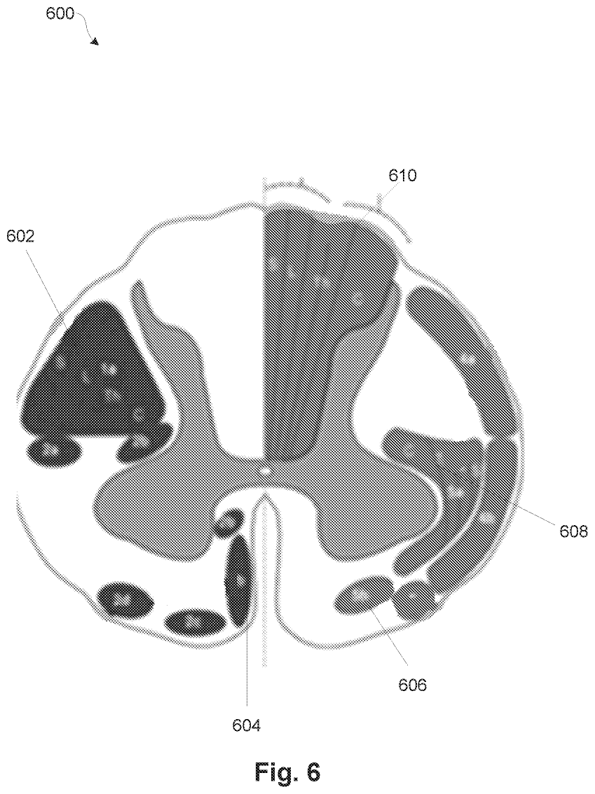

FIG. 6 illustrates a cross-section of the spinal cord 600 showing efferent pathways 602, 604 and afferent pathways 606, 608, 610. In the dorsoventral configuration of the described embodiments, probe measurement electrodes are positioned on the ventral (lower in FIG. 6) side of the spinal cord to measure the neural responses in the efferent pathways 604, and probe stimulus electrodes on the dorsal side (upper in FIG. 6) to recruit the afferent pathways 610 for H-wave evocation.

The following steps 406 and 408 of method 400 utilize the probe stimulus and response data from the stimulation and measurement of (Ia) afferent and (α-MN) efferent fibres respectively to determine stretch reflex excitability of the muscle, as described at steps 402 and 404. With reference to FIG. 4, at step 406 the controller 116 processes the neural response intensity values R at the intensity values of the stimulus S to determine a response growth curve indicating a degree of excitability of the stretch reflex of the muscle.

In the described embodiments, the values of the H-wave response growth curve are determined from the H-reflex of the muscle, and therefore represent the interaction between the Ia afferents and α-MNs within the spinal cord, as described above. The H-reflex is characterized at least by the presence of an evoked H-wave occurring as a function of the intensity of the stimulus applied to the Ia afferents. At low current intensities (below a H-wave motor threshold), no H-wave is produced. Once the stimulus intensity reaches the H-wave motor threshold, a H-wave (the reflex response or the monosynaptic H-reflex) is evoked in the α-MNs. In response to peripheral stimulation of the muscle, and for a stimulus intensity above an M-wave motor threshold, the α-MNs may be recruited directly, evoking an M-wave in the α-MNs.

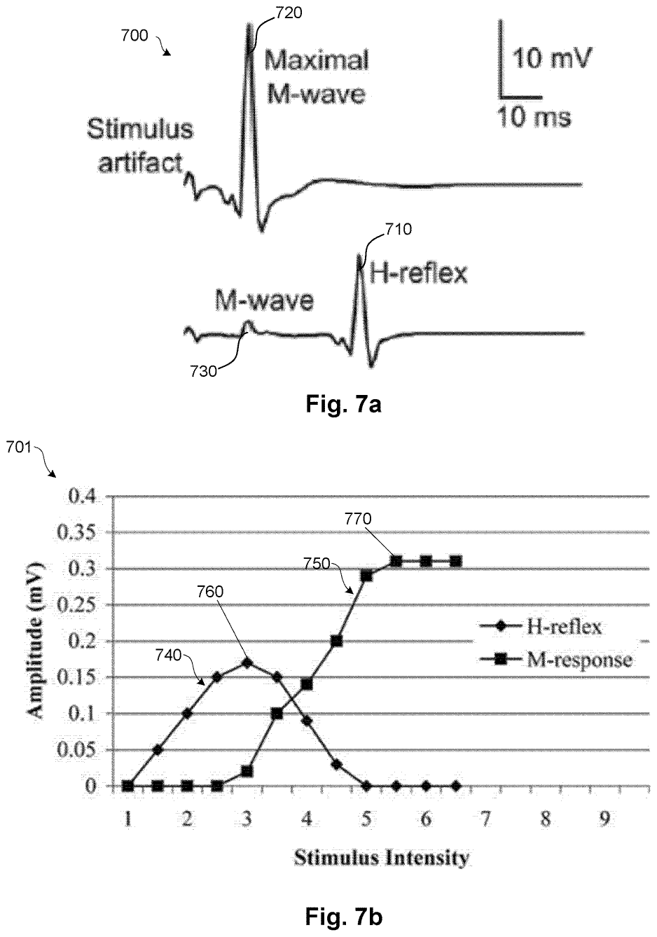

FIG. 7a is an illustration 700 (from Knikou [2]) of a H-wave 710 and M-waves 720, 730 evoked by the stimulation of nerves of the soleus muscle (i.e., from posterior tibial nerve stimulation) at the knee for applied probe stimulus current pulses of differing amplitudes.

FIG. 7b (from Palmieri [3]) is an illustration 701 of example response growth curves, including a response growth curve 740 of the H-wave and a corresponding response growth curve 750 of the M-wave for a peripheral muscular stimulus. The stimulus intensity values in FIG. 7b are normalized by the H-wave motor threshold. The H-wave amplitudes increase with increasing stimulus intensity above the H-wave motor threshold, until the stimulus intensity reaches the M-wave motor threshold and an M-wave is evoked, as indicated by the relatively small M-wave 730 preceding the H-wave 710 in FIG. 7a. As the stimulus intensity increases further, the H-wave reaches a maximum amplitude Hₘₐₓ 760. The H-wave amplitude then starts to decrease while the M-wave amplitude increases. For further increases to the stimulus intensity, the M-wave reaches a maximum amplitude Mₘₐₓ 770, as indicated by the maximal M-wave 720, which is not followed by a H-wave in FIG. 7a.

In embodiments in which the probe stimulus electrodes are located such that the M-wave is not evoked, the H-wave response growth curve does not peak and then decline as in FIG. 7b, but is sigmoidal in shape, rising to a maximum value as stimulus intensity increases above the H-wave motor threshold and saturating thereafter.

Processor 117 is configured, at step 406, to generate response growth curve data by processing the response data (i.e., ECAP values) obtained from the application of the probe stimuli. The H-wave response growth curve data C_{H} represents amplitude values of the H-wave evoked by the applied probe stimuli over intensity set S. In embodiments in which an M-wave is evoked, a corresponding M-wave response growth curve C_{M} is generated representing the amplitude values of the corresponding M-wave. The H-wave and M-wave values are determined from response values measured from the α-MNs (efferent fibres) at step 404. Each curve C_{H}, C_{M} is defined by data values that represent the respective H- and M-waves of the α-MNs for progressively increasing values of the applied stimulus intensity.

At step 408, an excitation response (ER) value is determined from the H-wave response growth curve C_{H}, and (in some embodiments) the corresponding M-wave response growth curve C_{M}.

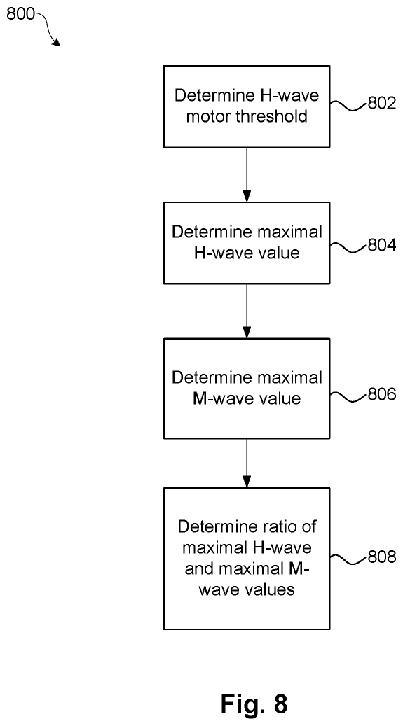

FIG. 8 illustrates a method 800 for determining the ER value based on determining the maxima of the response characteristics of both the H-wave and the M-wave over the range of stimulus intensities according to one embodiment. At step 802, controller 116 processes the H-wave growth curve data C_{H} to determine the intensity value Sₘₒₜₒᵣ corresponding to the H-wave motor threshold. In some embodiments, the stimulus intensity values Sᵢ are normalized by the Sₘₒₜₒᵣ value to generate normalized intensity values

$${S}_{i}^{norm} = {S}_{i} / {S}_{motor} .$$

At step 804, the controller 116 determines a maximal response value Hₘₐₓ of the evoked H-wave. A maximum function is applied to the values of the H-wave response growth curve data C_{H} generated for stimulus intensities above the H-wave motor threshold Sₘₒₜₒᵣ to determine Hₘₐₓ as a recorded response intensity (i.e., data point). In other embodiments, the controller 116 is configured to estimate the value of Hₘₐₓ by execution of an interpolation function on the determined H-wave response growth curve data C_{H}.

The maximal M-wave response value Mₘₐₓ is then determined at step 806. In some embodiments, the processor 117 is configured to determine the maximum M-wave amplitude Mₘₐₓ from the M-wave response growth curve C_{M}. In other embodiments, the controller 116 is configured to be programmed with the maximal M-wave response value Mₘₐₓ. The programmed value may be an average value obtained from the patient 108, or other individuals, in prior conducted clinical evaluations of the muscle or muscle group.

At step 808, the ER value is determined representing a characteristic degree of excitability of the stretch reflex based on the maximum amplitudes of the H-wave and the M-wave, Hₘₐₓ and Mₘₐₓ. The processor 117 is configured to calculate the ER value as the ratio of the values Hₘₐₓ and Mₘₐₓ, such that ER=Hₘₐₓ/Mₘₐₓ. The ER value thereby provides a measure of stretch reflex excitability that accounts for the variability in M-wave measurements between individuals.

In embodiments in which the probe stimulus electrodes are located such that there is no evoked M-wave and therefore no M-wave response growth curve C_{M}, the processor 117 is configured to determine the ER value from the (sigmoidal) H-wave response growth curve C_{H} alone at step 408. In such embodiments, the ER value may be determined as one of various properties of the H-wave response growth curve C_{H}, such as: the H-wave motor threshold Sₘₒₜₒᵣ; the maximum slope of the H-wave response growth curve C_{H}; the H-wave response value corresponding to a predetermined multiple (e.g. 1.2) of the H-wave motor threshold Sₘₒₜₒᵣ; or the maximal H-wave value Hₘₐₓ.

### Determining the RTL

With reference to FIG. 4, at step 410 the determined ER value is processed to generate the RTL for use as a target value in closed-loop control of SCS to relieve the muscle spasticity. In the described embodiments, processing of the determined ER value is performed “on-device” by the controller 116 of stimulator 100, such as by the execution of one or more control programs 122. In other embodiments, the stimulator 100 is configured to transmit the ER value to the user device 192, or another similar computing device, including a processor that is configured to perform the processing steps described below for RTL generation.

In some embodiments, the controller 116 is configured to generate the RTL by mapping the determined ER value to a corresponding target value, such as an ECAP value or another electrical field parameter value that is measurable by a system configured to perform closed-loop SCS to relieve spasticity in the muscle.

In other embodiments, the controller 116 is configured to generate the RTL by applying therapeutic stimuli of varying intensities, measuring the neural response intensities evoked by the therapeutic stimuli, and simultaneously and repeatedly determining ER values. When the determined ER value reaches an acceptable level, based on all available data, the neural response intensity is recorded as the RTL. The therapeutic stimuli of varying intensities may be applied in closed-loop fashion, to a variable target ECAP value, or open-loop fashion. In the closed-loop case, the target ECAP value when the determined ER value reaches an acceptable level is recorded as the RTL.

### Predetermined Table of Validated ECAP Values (Prior Clinical Evaluation)

In one implementation, the controller 116 retrieves the RTL from a response table stored in local memory (e.g., as part of a control program 122). The response table includes target ECAP values V (“validated ECAP values”) for each of a series of candidate ER values ER ¿. The validated ECAP values represent, for a candidate ER value representing a degree of stretch reflex excitability in the given muscle, a measured neural response intensity of the minimum therapeutic stimulus intensity required to relieve the spasticity in the muscle, when the therapeutic stimulus is applied to inhibit the stretch reflex by SCS in a validation test.

The validation tests performed to acquire the validated ECAP values and candidate ER values include one or more experimental trials conducted on an evaluation group of patients experiencing spasticity in the given muscle. For an evaluation group consisting of P patients with common spasticity in the given muscle, steps 402 to 408 are performed for each patient to obtain sample candidate ER values ER₁, . . . . ER_{P}. Conducting SCS on each patient of the evaluation group with an evaluation stimulus of increasing intensity results in the measurement of a corresponding set of neural response intensity values. A clinician assesses the muscle spasticity in each evaluation patient as a function of the evaluation stimulus intensity and determines the minimum stimulus intensity level for which spasticity is relieved. The corresponding response intensity is selected as the validated ECAP value V and entered into the response table in association with the candidate ER value of the evaluation patient.

In some embodiments, the user device 192 is configured to enable the determination of candidate ER and validated ECAP values, and to derive the response table for determining an RTL for closed-loop SCS. The values may be stored by the processor 194 as clinical evaluation data within the memory system 196 of user device 192. An application program executing on the device 192 may be configured to generate corresponding response table values by extracting the stored evaluation data and performing arithmetic operations (e.g., to sort, exchange, or arrange the data in a list, array, hashtable, or other data structure). The user device 192 is configured to program the stimulator 100 with the derived response table by an exchange of data between the telemetry module 114 and the device 192 over the communication channel 190.

To determine the RTL using the programmed response table, the controller 116 determines the index of the recorded ER value ER₁, . . . . ER_{P} (e.g., the ER values of the respective evaluation group patients) within the table that is closest to the determined ER value ER_{d} generated for patient 108. In one implementation, the processor calculates the index by determining the Euclidean distance between the respective values

$$( i . e . , as j = arg {min}_{i} ( \vert {ER}_{i} - {ER}_{d} \vert ) ∀ i = 1 … P ) .$$

The corresponding validated ECAP value (Vⱼ) is retrieved from the response table and set as the RTL. The contents of the response table as stored by the controller 116 may be set and/or updated by a transmission of table update data to the controller 116 from the user device 192. The table update data may be transmitted periodically or in an ad hoc manner to facilitate the ability of the stimulator 100 to determine an RTL that enables effective spasticity relief for the given muscle.

In another implementation, the controller may interpolate between the two validated ECAP values Vⱼ corresponding to the recorded ER values that straddle the determined ER value ER_{d} to obtain the RTL.

Prediction from Response Model and ER Differential

FIG. 9 illustrates a method 900 for determining the RTL from an ER value using a response model. The response model is trained on ER and corresponding ECAP value data obtained prior to step 404, such as for example as a result of a clinical trial or evaluation performed on an evaluation group of patients with spasticity in the given muscle (as described above for response table generation). The RTL is obtained as a target ECAP value that is predicted or output from the application of the response model to an input ER value (i.e., the determined ER value for patient 108).

The ER value (e.g., the Hₘₐₓ/Mₘₐₓ ratio) is a quantification of the excitability of the stretch reflex of an individual and therefore represents the degree to which movement (inhibition) is compromised in the given muscle. That is, the determined value ER_{d} for patient 108 is contingent on the lack of descending α-MN inhibition and therefore the degree or extent to which spasticity presently afflicts the given muscle. Furthermore, there is also an assumed monotonic relationship between the intensity of an applied stimulus and the measured response value (see FIG. 3b). Based on these relationships, response models can be constructed to relate the extent to which the ER value of patient 108 (ER_{d}) exceeds the expected value of healthy individuals (ER), to an intensity of applied therapeutic stimulus effective to overcome the lack of inhibition (and therefore a measured response intensity value, or other electrical field parameter value, to set as the RTL).

In the described embodiments, the response model is applied to an ER differential value calculated as ΔER=ER_{d}−E̅R̅ where ER_{d} is the ER value determined for patient 108 and E̅R̅ is the average ER value of healthy individuals (i.e., with no spasticity in the muscle). The average ER value is determined by a training program executed on the user device 192. ER values are obtained representing determinations of the excitability of the stretch reflex in the given muscle across a control group of individuals for whom the given muscle is not afflicted with spasticity. A set of ER values is obtained by the user device 192 via a data exchange between respective stimulator devices 100 of the control group individuals. Each stimulator 100 of a control group individual applies the steps 402-408 to determine a candidate ER value for the expected stretch reflex excitability of the muscle. The ER values are transmitted from each stimulator 100 to the user device 192. The user device 192 averages the candidate values to determine the expected ER value E̅R̅.

The user device 192 may be configured to store the expected ER value E̅R̅ as part of clinical evaluation data, or related data, in the memory system 196. Note that the configuration of stimulator 100 may differ in healthy individuals of the control group compared to spasticity afflicted patients, such as patient 108. For example, stimulator 100 may be located cutaneously such that neither control module 110 nor electrode array 150 are implanted within the healthy individual.

In embodiments where the RTL for patient 108 is determined by the stimulator 100, the user device 192 is configured to program the stimulator 100 with the expected ER value E̅R̅. The processor 194 retrieves the expected ER value from the data of memory system 196 and transmits the value to the controller 116 via a data exchange over the communications channel 190. At step 902, the controller 116 retrieves the expected ER value E̅R̅ from local memory for use by a control program 122.

At step 904, the processor 117 calculates the ER differential value ΔER by subtracting the expected ER value ER from the determined ER value of patient 108 ER_{d}. At step 906, controller 116 determines the RTL as a target ECAP value by applying a response model to the ER differential value. One or more parameters defining the response model are stored in the memory of controller 116, for example as part of the one or more control programs 122 or associated data.

In some implementations, the response model is a linear regression model such that the magnitude of the target ECAP value to be used as the RTL is modelled as linearly related to the ER differential value. Parameters of the linear regression model are trained on a set of data points D obtained from trials on an evaluation group of patients with spasticity afflicting the given muscle, as described above. The controller 116 of the stimulator 100 is programmed with the model parameters following training via a data exchange with user device 192.

For example, a training set of m samples D={d₁, . . . , dₘ} may include a data point dᵢ=(ΔERᵢ=ERᵢ−E̅R̅, Vᵢ) for each evaluation patient i. In one form, the response model is a linear predictor function T (ΔER)=α+β. ΔER, where output T(.) is the RTL corresponding to the input ER differential, and value of the α and β coefficients are determined by performing a regression training process on the evaluation training data set D (e.g., least-squares fit). The controller 116 is configured to apply the response model to the ER differential value determined for patient 108, ΔER_{d}, to calculate the RTL (i.e., as T (ΔER_{d})) by executing a corresponding routine or function of a control program 122 with the programmed model parameters (i.e., α and β).

Alternatively, or in addition, the response model includes a set of classification parameters λ such that the target ECAP value is output from a pattern classifier operating on λ and an input of the ER differential value (e.g., ΔER_{d} of patient 108).

For example, the response model may include parameters describing a neural network (NN) classifier, including, for example, a feedforward neural network, a multilayer perceptron, or a convolutional neural network (CNN). In one implementation, the NN is configured as a single-layer feedforward network with one input corresponding to a ΔER value and one output T corresponding to the predicted target ECAP value or field parameter measurement (the RTL). The model parameters include one or more weights λ=(α₁, . . . , α_{w}) of w nodes of at least one hidden layer. Supervised training of the model parameters is performed using a training set of m samples D={d₁, . . . , dₘ} as described above. In some embodiments, a backpropagation process is employed during training to adjust the node connection weights to compensate for errors (e.g., based on a predetermined cost function).

In some embodiments, the feedforward NN is configured as a multi-layer network and/or with multiple input variables. For example, the network may be configured with a 2-dimensional or higher input layer that accepts data values of the determined ER differential ΔER and one or more other variables. This enables a modelling of the desired RTL value based on the collective knowledge of the stretch reflex excitability in conjunction with other characteristics of the patient and/or the muscle (e.g., patient age, height, and/or weight, muscle size, etc.). The controller 116 of the stimulator 100 is programmed with the model parameters λ following NN training via a data exchange with user device 192. The controller 116 is configured to calculate the RTL by executing a corresponding NN evaluation routine with the programmed model parameters (i.e., λ), and using at least the determined ER differential of patient 108 ΔER_{d} as an input to the routine.

In some embodiments, the application of a response model to the ER differential value is performed partly or wholly by the user device 192. For example, the user device 192 may be configured to perform one or more of steps 904 and 906 by the execution of one or more application programs with data received from the stimulator 100 of patient 108 (e.g., including at least the determined ER value ER_{d}, if not already obtained by the device 192). In some embodiments, the determined ER value ER_{d} is used directly, without the determination of a corresponding ER differential value, to calculate the RTL (e.g., by training the response model on corresponding ER values of evaluation group patients).

In some embodiments, the calculation of the RTL is performed by a processor 117, 194 firstly applying one or more pre-processing and/or correction functions to the training data and/or the determined ER value for patient 108. In some embodiments, the processor 117, 194 determines the ER value for patient 108 from a set of multiple sample ER values, such as for example over successive repetitions of steps 402-408, before proceeding to perform step 408 with an aggregated, expected, or averaged value of the sample ER values.

### Performing Closed-Loop SCS

Referring to FIG. 3a, determination of the RTL at step 304 enables a neuromodulation device, such as stimulator 100, to perform closed-loop SCS therapy to relieve muscle spasticity in the patient 108 (i.e., at step 306).

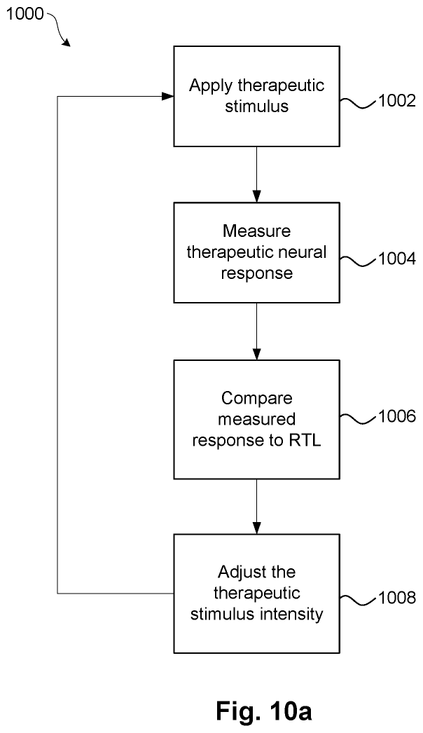

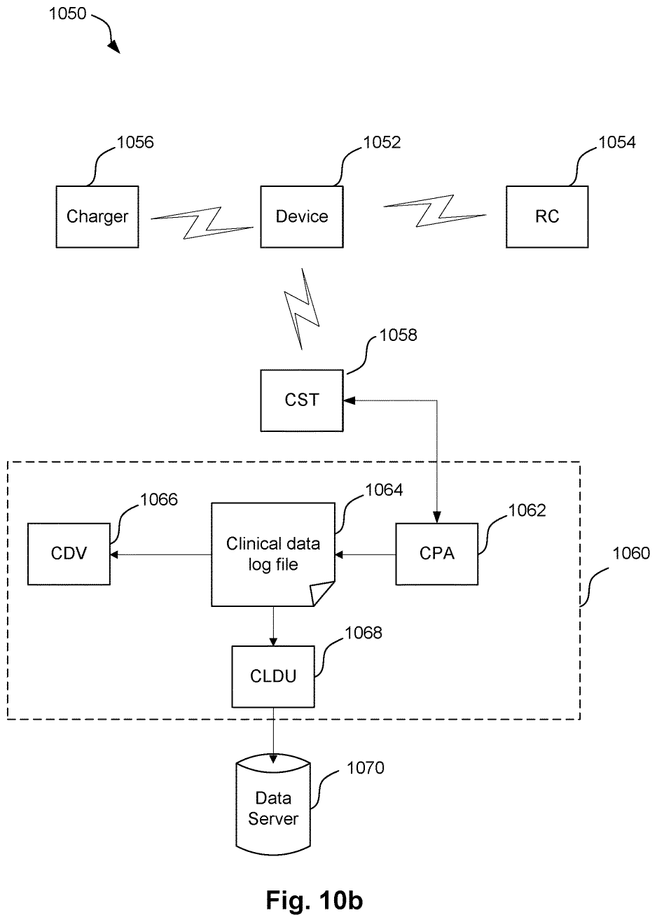

FIG. 10a illustrates a method 1000 of performing closed-loop SCS therapy via a neuromodulation device as a treatment to relieve muscle spasticity in the patient 108. FIG. 10b is a block diagram of a neuromodulation system 1050 configured to perform the method 1000. In one example, the neuromodulation device 1052 of FIG. 10b is implemented as the stimulator 100 of FIG. 1, implanted within the patient 108 (not shown). In this mode of operation, stimulator 100 is a Closed-Loop Neural Stimulation (CLNS) device.

The neuromodulation device 1052 is connected wirelessly to a remote controller (RC) 1054. The remote controller 1054 is a portable computing device that provides the patient 108 with control of the closed-loop SCS therapy by providing selective control over at least some of the functionality of the neuromodulation device 1052, including: enabling or disabling closed-loop SCS according to a spasticity relief program; and selection of a spasticity relief program from the control programs stored on the neuromodulation device 1052 (e.g., control programs 122 of stimulator 100).

In some embodiments, the spasticity relief programs of the neuromodulation device 1052 are configured by an external computing device, such as the user device 192, and are uploaded to the neuromodulation device 1052 by a user or clinician. In some embodiments, the neuromodulation device 1052 is configured to commence, continue and/or cease therapeutic closed-loop SCS autonomously and independently of instructions or signals received from the user device 192, or any other computing device, of the therapy system 1050.

Neuromodulation system 1050 includes a charger 1056 configured to recharge a rechargeable power source of the neuromodulation device 1052. The recharging is illustrated as wireless in FIG. 10b but may be wired in alternative implementations.

The neuromodulation device 1052 is wirelessly connected to a Clinical System Transceiver (CST) 1058. The wireless connection may be implemented as the transcutaneous communications channel 190 of FIG. 1. The CST 1058 acts as an intermediary between the neuromodulation device 1052 and a Clinical Interface (CI) 1060, to which the CST 1058 is connected. A wired connection is shown in FIG. 10b, but in other implementations, the connection between the CST 1058 and the CI 1060 is wireless.

The CI 1060 may be implemented as the user device 192 of FIG. 1. The CI 1060 is configured to program the neuromodulation device 1052 and recover data stored on the neuromodulation device 1052. This configuration is achieved by program instructions collectively referred to as the Clinical Programming Application (CPA) 1062 and stored in an instruction memory of the CI 1060. For example, the CPA may specify particular settings and/or operational modes of the neuromodulation device 1052 according to the context of the therapeutic spasticity relief that is to be provided by the system 1050. For example, some applications of the system 1050 relate to relieving spasticity as part of post-stroke rehabilitation. In such applications, specific therapy settings may be chosen to, for example, account for an increased difficulty in obtaining qualitative feedback from the patient with respect to the treatment.

To effect suitable SCS therapy, neuromodulation device 1052 may deliver tens, hundreds or even thousands of therapeutic stimuli per second, for many hours each day. The feedback loop may operate for most or all of this time, by obtaining neural response recordings following every therapeutic stimulus, or at least obtaining such recordings regularly. Each recording generates a feedback variable such as a measure of the amplitude of the evoked neural response, which in turn results in the feedback loop changing at least one stimulus parameter for a following therapeutic stimulus. Neuromodulation device 1052 thus produces such data at a rate of tens or hundreds of Hz, or even kHz, and over the course of hours or days this process results in large amounts of clinical data. This is unlike past neuromodulation devices such as open-loop SCS devices which lack any ability to record any neural response.

When brought in range with a receiver, neuromodulation device 1052 transmits data, e.g. via telemetry module 114, to a CPA 1062 installed on the CI 1060. The data can be grouped into two main sources: (1) Data collected in real time during a programming session; (2) Data downloaded from a stimulator after a period of non-clinical use by a patient. The CPA collects and compiles the data into a clinical data log file.

All clinical data transmitted by the neuromodulation device 1052 may be compressed by use of a suitable data compression technique before transmission by telemetry module 114 and/or before storage into the memory 118 to enable storage by neuromodulation device 1052 of higher resolution data. This higher resolution allows neuromodulation device 1052 to provide more data for post-analysis and more detailed data mining for events during use. Alternatively, compression enables faster transmission of standard-resolution clinical data.

The clinical data log file 1064 is manipulated, analysed, and efficiently presented by a clinical data viewer (CDV) 1066 for field diagnosis by a clinician, field clinical engineer (FCE) or the like. CDV 1066 is a software application installed on the CI 1060. In one implementation, CDV 1066 opens one Clinical Data Log file 1064 at a time. CDV 1066 is intended to be used in the field to diagnose patient issues and optimise therapy for the patient. CDV 1066 may be configured to provide the user or clinician with a summary of neuromodulation device usage, therapy output, and errors, in a simple single-view page immediately after log files are compiled upon device connection.

Clinical Data Uploader (CLDU) 1068 is an application that runs in the background on the CI 1060, that uploads files generated by the CPA 1062, such as the clinical data log file 1064, to a data server 1070. Database Loader (not shown) is a service which runs on the data server and monitors the patient data folder for new files. In response to Clinical Data Log file 1064 being uploaded by Clinical Data Uploader 1068, a database loader extracts the data from the file and loads the extracted data to a database of the data server 1070 (not shown).

The data server 1070 further contains one or more APIs, such as for example a data analysis web API, which provide data for third-party analysis such as by one or more computing devices located remotely from the data server 1070. The ability to obtain, store, download and analyse large amounts of neuromodulation data in accordance with the methods described herein is advantageous in: improving patient outcomes in difficult conditions; enabling faster, more cost effective and more accurate troubleshooting and patient status; and enabling the gathering of statistics across patient populations for later analysis, with a view to diagnosing aetiologies and predicting patient outcomes.

Referring to FIG. 10a, in the described embodiments the method 1000 for closed-loop SCS treatment to relieve spasticity of a muscle is performed by stimulator 100 and user device 192, as depicted in FIG. 1. In other embodiments, another CLNS device may be configured to perform the method 1000. At step 1002, stimulator 100 applies a therapeutic stimulus of an initial intensity to the spinal cord to stimulate the one or more Aβ (afferent) fibres associated with the muscle as a treatment to relieve the spasticity of the muscle. Pulse generator 124 applies the therapeutic stimulus to the dorsal column via therapeutic electrodes of array 150. The therapeutic electrodes may be positioned rostrally (closer to the brain) along the dorsal column in relation to the probe stimulus and probe measurement electrodes. The initial therapeutic stimulus intensity value is specified by a control program 122 such as a closed-loop therapy routine with patient-specific and/or therapy-specific parameters determined by the clinical settings 121.

At step 1004, the stimulator 100 measures an intensity of a therapeutic neural response evoked by the application of the therapeutic stimulus in the one or more Aβ fibres. The intensity of the evoked therapeutic neural response (e.g., the measured amplitude of the evoked neural response signal) provides a measure of the recruitment of the fibres being stimulated. A monotonic response curve between the applied therapeutic stimulus intensity and the evoked therapeutic neural response intensity is assumed as shown in FIG. 3b. The therapeutic neural response intensity values are measured by measurement circuitry 128 from an electrical signal sensed by therapeutic electrodes of electrode array 150. In one implementation, the therapeutic neural response intensity comprises a peak-to-peak ECAP amplitude.

The measured therapeutic neural response intensity values are processed by the controller 116 which performs closed-loop modulation of the therapeutic stimulus to relieve the spasticity of the muscle (i.e., by ensuring an appropriate therapeutic stimulus intensity to compensate for the lack of inhibition of the α-MN fibres). The controller 116 generates a feedback signal representing a difference between values of the therapeutic neural response intensity r and the RTL determined for patient 108.

At step 1006, the controller 116 compares the measured therapeutic neural response intensity to a target ECAP value (the RTL) and provides an indication of the difference as an error value. The error value is input into a feedback unit of the controller 116. At step 1008, the feedback unit of controller 116 calculates an adjusted therapeutic stimulus intensity parameter with the aim of maintaining a measured therapeutic neural response intensity equal to the RTL. The feedback unit of controller 116 adjusts the therapeutic stimulus intensity parameter to minimise the error value. In one implementation, the controller 116 utilises a first order integrating function in order to provide suitable adjustment to the therapeutic stimulus intensity parameter. In some embodiments, the feedback unit of controller 116 may be configured to adjust the therapeutic stimulus intensity based on one or more other settings or parameters. For example, the feedback unit may be configured to a set a gain parameter to generate the feedback signal based on the clinical settings 121, such as to account for patient specific tolerances and/or sensitivities.

The controller 116 is configured to repeatedly apply therapeutic stimuli at the adjusted intensity values to achieve closed-loop control of the therapeutic stimulation of the Aβ fibres (i.e., by repeating step 1002 following step 1008). In some implementations, the re-application of the therapeutic stimulus with the adjusted stimulus intensity is controlled by a stimulus clock operating at a stimulus frequency (e.g. 60 Hz) and a sample clock for sampling the therapeutic neural response signal (for example, operating at a sampling frequency of 10 kHz). On the next stimulus clock cycle, the stimulator 100 outputs a therapeutic stimulus in accordance with the adjusted therapeutic stimulus intensity. Accordingly, there is a delay of one stimulus clock cycle before the therapeutic stimulus intensity is updated in light of the error value.

### Assessing a Therapy for Spasticity Relief

FIG. 11 is a block diagram of a method 1100 of assessing a treatment to relieve spasticity in the given muscle of patient 108. Referring to FIG. 3a, in some embodiments the assessment is performed, at step 308, to evaluate the efficacy of a closed-loop SCS spasticity relief program executed by the neuromodulation system 1050 for the given muscle of patient 108 (i.e., at step 306). That is, the neuromodulation system 1050 may be configured to perform both the closed-loop SCS therapy and a subsequent assessment of the therapy as a muscle-specific spasticity relief treatment.

In other embodiments, the neuromodulation system 1050 is configured to perform the steps of method 1100 to evaluate another treatment, such as closed-loop SCS performed by another CLNS device or neuromodulation therapy system, or open-loop SCS, or a non-SCS-based treatment (e.g., the administration of systemic agents to reduce hyper-excitability, such as benzodiazepines and baclofen, or surgical ligation of the Ia afferent nerve).

To perform the assessment, the neuromodulation system 1050 determines an excitation response (ER) value to characterize the present degree of excitability of the muscle stretch reflex by performing steps 1102 to 1108 of method 1100. In the described embodiments, the neuromodulation device 1052 and CI 1060 of the system 1050 are implemented as the stimulator 100 and user device 192 respectively of FIG. 1, and the steps 1102 to 1108 are performed analogously to steps 402 to 408 as described above with reference to FIG. 4. The assessment method 1100 may be performed when the patient is relaxed, i.e. has no intention to move the spastic muscle group, to minimise the chance of any residual descending inhibition from the brain contaminating the assessment.

At step 1110, the determined ER value ER_{d} for patient 108 is compared to one or more other ER values associated with the treatment to relieve the spasticity of the muscle. The comparison is performed by a processor of the neuromodulation system 1050, such as processor 117 or 194 depending on whether the assessment is performed on the neuromodulation device (e.g., by the controller 116 of the stimulator 100), or by an external computing device (e.g., the user device 192).

In some embodiments, the one or more other ER values define an expected range of ER values for the given muscle without spasticity. By comparing the ER_{d} value to the one or more other values the controller 116 may determine, at step 1112, a relative degree of spasticity of the given muscle. For example, controller 116 may be configured to retrieve a pair of ER values ERₘᵢₙ, ERₘₐₓ defining a range or interval for the expected, average, or normal excitation response of the muscle without spasticity. The controller 116 may provide a binary indication of spasticity within the muscle (i.e., whether ER_{d} is within the interval), and/or provide an indication of a degree of the spasticity condition based on the difference between the ER_{d} value and a representative value of the interval. In another example, a single ER value ERₜ may be retrieved by controller 116 for use as a threshold to determine whether spasticity exists in the given muscle (i.e., where positive verification of spasticity occurs if ER_{d}>ERₜ).

The assessment of a spasticity treatment is based on the ability of a set of ER values to indicate corresponding degrees of excitability of the stretch reflex, and to therefore enable the inference of a degree of hyper-excitability (which is characteristic of spasticity) that presently exists for the muscle, and a progression, or change, in that state over time. In one embodiment, steps 1102 to 1108 are repeated to generate a series of determined ER values ER_{d1}, . . . , ER_{dT} at corresponding time instants t₁, . . . , t_{T}. Each determined ER value ER_{di} for i=1, . . . , T may correspond to a measurement of the excitation response obtained following the i-th application of one iteration, or application of a particular treatment completing at a time tᵢ (e.g., the execution of a spasticity relief routine by the neuromodulation system 1050).

At step 1112, the series of ER determined values ER_{d1}, . . . , ER_{dT} are processed, either by processor 117 of controller 116 or processor 194 of the user device 192, to monitor the relative degree of spasticity of the given muscle over time.

In one example, the controller 116 is configured to record a series of one or more ER values ER_{d1}, . . . , ER_{dT} determined from previously performed therapeutic operations of the stimulator 100. The ER values are stored within local memory of a control program 122, or as part of the clinical data 120 or clinical settings 121. The controller 116 is configured to assess the treatment by comparing a presently determined ER value ER_{d} to the one or more other historical (previously determined) ER values ER_{d1}, . . . , ER_{dT}.

For example, the controller 116 may be configured to calculate the difference between the ER_{d} value and the mean, median, or other representative measure of the N≥1 previously determined ER values as

$${ER}_{d} - {\sum }_{i = 0}^{N - 1} {ER}_{d ( T - i )} / N .$$

A presently determined value ER_{d} that is less than the representative value of the previously determined ER values (i.e., a negative difference) may indicate a reduced degree of hyper-excitability, and therefore an effective treatment.

Various other approaches may be implemented to assess spasticity treatments based on a series of determined ER values ER_{d1}, . . . , ER_{dT} representing a degree of hyper-excitability of the stretch reflex of a given muscle over time t₁, . . . , t_{T}. For example, statistical analysis may be conducted on the data values ER_{d1}, . . . , ER_{dT} to infer a temporal relation or trend in the values over time. The determined ER values ER_{d1}, . . . , ER_{dT} may be used to construct a visual representation (e.g., a 2D plot or surface curve) of stretch reflex excitability enabling a clinician to form a qualitative assessment on the state of the spasticity condition and/or the effectiveness of one or more therapies resulting in the determined ER values. In one or more of the approaches, processing of the determined ER values ER_{d1}, . . . , ER_{dT} is performed by the user device 192, following the transmission of the determined ER values to the user device 192 (e.g., as clinical data) by the stimulator 100.

In some embodiments, the controller 116 of the stimulator 100 is configured to transmit the determined ER values to the user device 192 irrespective of which device performs the processing associated with the assessment of therapy. This enables the user device 192 to store the determined ER values and thereby maintain a historical record (e.g., as data entries in a table or database) of assessments of spasticity relief for patient 108.

Referring to FIG. 3a, assessment of a spasticity treatment may be performed by the neuromodulation system 1050 within an integrated approach to closed-loop SCS therapy (step 306) in which an initially determined feedback target (i.e., RTL value at step 304) is dynamically adjusted based on the assessment outcome (at step 308). In one embodiment, the aforementioned steps form an “outer” closed-loop where the RTL value used for the SCS spasticity therapy (which itself has an “inner” closed-loop) is continuously adjusted based on the assessment of the SCS spasticity therapy (e.g., the difference in the ER value presently produced relative to a historical representative value, as described above).

In another embodiment, processor 117 of the controller 116 (or processor 194 of the user device 192) initiates an update to the RTL value used for performing closed-loop SCS, by causing the neuromodulation system to repeat step 304, in response to a particular assessment outcome. For example, an RTL update may be caused in response to the degree of spasticity (as quantified by the ER_{d} value) falling by a predetermined amount since the last update to the RTL value. In some embodiments, ER values determined in particular steps of integrated method 300, such as the RTL determination at step 304, are buffered, or otherwise stored locally in the controller 116 or the user device 192, to avoid the need to recalculate the same ER values in one or more subsequently performed steps (e.g., during assessment at step 308).

In another embodiment, the ER value itself may be determined during therapy and compared with a desired ER value or range to provide a feedback signal to adjust the stimulus intensity, thereby removing the need to measure the ECAP resulting from the therapeutic stimuli. In effect, the loop would be closed on the ER value rather than the therapeutic neural response intensity. This advantageously enables the neuromodulation system 1050 to control the applied therapeutic stimulus intensity directly from the reflex excitability (i.e., the determined ER value) without explicit measurement or processing of the corresponding therapeutic neural response intensity.

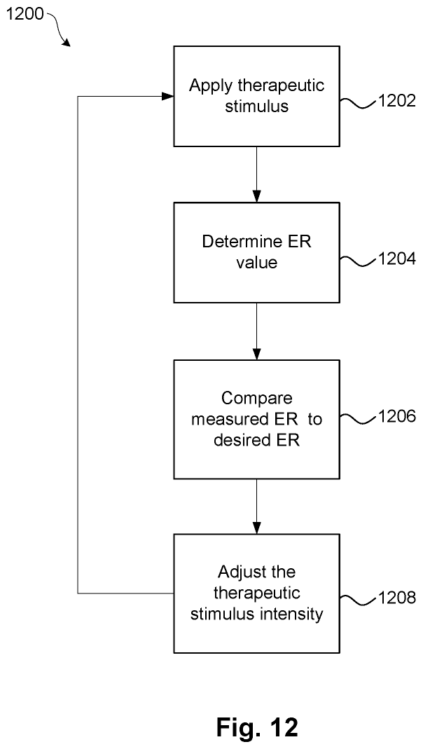

FIG. 12 illustrates a method 1200 of performing closed-loop SCS therapy via a neuromodulation device for the treatment of muscle spasticity in the patient 108 according to this embodiment. The method 1200 may be performed by the neuromodulation system 1050 of FIG. 10b.

At step 1202, stimulator 100 applies a therapeutic stimulus of an initial intensity to the spinal cord to stimulate the one or more Aβ fibres. At step 1204, the controller 116 determines the current ER value, as in step 408 of the method 400. At step 1206, the controller 116 compares the determined ER value to a desired ER value, or a desired ER range, and provides an indication of the difference as an error value. The error value is input into a feedback unit of the controller 116. At step 1208, the feedback unit of controller 116 calculates an adjusted therapeutic stimulus intensity parameter with the aim of maintaining the determined ER value equal to the desired ER value, or within the desired ER range. The method 1200 then returns to step 1202 to apply the next therapeutic stimulus (i.e., of the adjusted intensity). Such embodiments give more direct control over the desired outcome of the SCS therapy than those in which the therapy is mediated by the RTL. However, the determination of the ER value requires multiple probe stimuli, so the frequency of updating the therapeutic stimuli would be less than in a conventional CL-SCS system, or the probe stimuli would need to be delivered more frequently than the therapeutic stimuli. In addition such embodiments require the probe stimulus and measurement electrodes to be permanently implanted along with the therapeutic electrodes.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not limiting or restrictive.

LABEL LIST

| | |
|---|---|
| stimulator | 100 |
| patient | 108 |
| control module | 110 |
| battery | 112 |
| telemetry module | 114 |
| controller | 116 |
| processor | 117 |
| memory | 118 |
| clinical data | 120 |
| clinical settings | 121 |
| control programs | 122 |
| pulse generator | 124 |
| electrode selection module | 126 |
| measurement circuitry | 128 |
| electrode array | 150 |
| communications channel | 190 |
| user device | 192 |
| processor | 194 |
| memory system | 196 |
| method | 300 |
| step | 302 |
| step | 304 |
| step | 306 |
| step | 308 |
| activation plot | 350 |
| constant slope | 352 |
| ECAP threshold | 354 |
| method | 400 |
| step | 402 |
| step | 404 |
| step | 406 |
| step | 408 |
| step | 410 |
| spinal reflex arc | 500 |
| muscle spindle | 503 |
| afferent fibres | 504 |
| efferents | 506 |
| inhibitory interneurons | 507 |
| skin mechanoreceptors | 508 |
| dorsal root entry zone | 510 |
| ventral roots | 512 |
| muscle | 520 |
| spinal cord | 600 |
| efferent pathway | 602 |
| efferent pathway | 604 |
| afferent pathway | 606 |
| afferent pathway | 608 |
| afferent pathway | 610 |
| illustration | 700 |
| illustration | 701 |
| H - wave | 710 |
| M - wave | 720 |
| M - wave | 730 |
| growth curve of H-wave | 740 |
| growth curve of M-wave | 750 |
| H-wave maximum amplitude | 760 |
| M-wave maximum amplitude | 770 |
| method | 800 |
| step | 802 |
| step | 804 |
| step | 806 |
| step | 808 |
| method | 900 |
| step | 902 |
| step | 904 |
| step | 906 |
| method | 1000 |
| step | 1002 |
| step | 1004 |
| step | 1006 |
| step | 1008 |
| neuromodulation system | 1050 |
| neuromodulation device | 1052 |
| remote controller (RC) | 1054 |
| charger | 1056 |
| Clinical System Transceiver | 1058 |
| Clinical Interface (CI) | 1060 |
| Clinical Programming | 1062 |
| Application (CPA) | |
| clinical data log file | 1064 |
| Clinical Data Viewer (CDV) | 1066 |
| clinical data uploader | 1068 |
| data server | 1070 |
| method | 1100 |
| step | 1102 |
| step | 1104 |
| step | 1106 |
| step | 1108 |
| step | 1110 |
| step | 1112 |
| method | 1200 |
| step | 1202 |
| step | 1204 |
| step | 1206 |
| step | 1208 |

### REFERENCES

[1] J. Parker & B. Dietz, Spinal cord stimulation for the relief of spasticity from cerebral palsy, Healthcare Technology Letters, 2020-IET. 7(3) 93-97.
[2] M. Knikou, The H-reflex as a probe: Pathways and pitfalls. Journal of Neuroscience Methods 2008, 171 1-12.
[3] R. M. Palmieri et al., The Hoffmann reflex: methodologic considerations and applications for use in sports medicine and athletic training research, Journal of Athletic Training 2004, 39 (3) 268-277.

1. A method for determining a recruitment target level (RTL) for programming spinal cord stimulation (SCS) to relieve spasticity in a muscle, the method including:
controlling a stimulator device to apply probe stimuli of variable intensity to stimulate one or more afferent fibres of the muscle;
receiving values of intensity of neural responses measured via the stimulator device, the neural responses being evoked by respective probe stimuli in one or more efferent fibres of the muscle;
processing the neural response intensity values and the respective intensity values of the probe stimuli to determine a response growth curve indicating excitability of a stretch reflex of the muscle;
determining an excitation response (ER) value from the response growth curve; and
processing the ER value to generate the RTL as a target value for closed-loop control of the SCS.

2. The method of claim 1, further comprising receiving values of intensity of the applied probe stimuli measured via the stimulator device.

3. The method of claim 1, wherein the neural responses correspond to H-waves evoked in the one or more efferent fibres.

4. The method of claim 3, wherein the neural responses are evoked compound action potentials (ECAPs) in the one or more efferent fibres.

5. The method of claim 3, wherein the neural responses are EMGs in the one or more efferent fibres.

6. The method of claim 3, wherein the ER value is determined from a maximum slope of the response growth curve.

7. The method of claim 3, wherein the ER value is determined from a threshold of the response growth curve.

8. The method of claim 3, wherein the ER value is determined from a maximal amplitude of the evoked H-wave in the response growth curve.

9. The method of claim 8, wherein the ER value is further determined from a maximal amplitude of an evoked M-wave in the one or more efferent fibres.

10. The method of claim 9, wherein the ER value is determined as a ratio of the maximal amplitudes of the evoked H-wave and the evoked M-wave.

11. The method of claim 1, wherein generating the RTL comprises mapping the determined ER value to a corresponding target ECAP value.

12. The method of claim 11, wherein the target ECAP value is extracted from a predetermined response table including one or more candidate ER values and corresponding validated target ECAP values.

13. The method of claim 11, wherein the target ECAP value is determined by applying a response model to the determined ER value.

14. The method of claim 13, wherein the target ECAP value is determined by applying the response model to an ER differential value representing a difference between the determined ER value and an expected ER value of the muscle without spasticity.

15. The method of claim 13, wherein the response model is a linear regression model such that a magnitude of the target ECAP value is linearly proportional to the ER value.

16. The method of claim 13, wherein the response model is a set of classification parameters, such that the target ECAP value is output from a pattern classifier operating on the response model and an input of the ER value.

17. A method for performing spinal cord stimulation (SCS) to relieve spasticity of a muscle, the method including:
applying a therapeutic stimulus to the spinal cord to stimulate one or more afferent fibres associated with the muscle;
measuring, in response to the application of the therapeutic stimulus, an intensity of a therapeutic neural response evoked by the therapeutic stimulus in the one or more afferent fibres; and
adjusting an intensity of a subsequent therapeutic stimulus based on a feedback signal representing a difference between the measured therapeutic neural response intensity and an RTL determined by the method of claim 1.

18. The method of claim 17, wherein the stimulator device conducts the applying, measuring, and adjusting to perform SCS to relieve spasticity in response to being programmed with the determined RTL.

19. The method of claim 17, further including adjusting the RTL before applying a subsequent therapeutic stimulus.

20. The method of claim 19, wherein adjusting the RTL comprises repeating the controlling, receiving, processing and determining to determine an updated excitation response (ER) value.

21. The method of claim 20, wherein adjusting the RTL further comprises:
comparing the determined ER value to one or more other ER values associated with the treatment to relieve the spasticity of the muscle; and
adjusting the RTL based on the comparison.

22. A system for determining a recruitment target level (RTL) for programming spinal cord stimulation (SCS) to relieve spasticity in a muscle, the system including:
a stimulator device comprising an electrode array and a pulse generator, the stimulator device configured to:
apply, via the pulse generator, probe stimuli to stimulate one or more afferent fibres of the muscle via one or more probe stimulus electrodes of the electrode array; and
measure, via one or more probe measurement electrodes of the electrode array, values of intensity of neural responses evoked by respective probe stimuli in one or more efferent fibres of the muscle, and
a processor configured to:
control the stimulator device to apply the probe stimuli at variable intensity and measure corresponding values of neural response intensity;
process the neural response intensity values and the respective intensity values of the probe stimuli to determine a response growth curve indicating excitability of a stretch reflex of the muscle;
determine an excitation response (ER) value from the response growth curve; and
process the ER value to generate the RTL as a target value for closed-loop control of the SCS.

23. The system of claim 22, wherein the probe stimulus electrodes are implanted adjacent to the afferent fibres on the dorsal side of the spinal cord.

24. The system of claim 22, wherein the probe stimulus electrodes are implanted adjacent to the afferent fibres at a dorsal root of the spinal cord.

25. The system of claim 22, wherein the probe stimulus electrodes are located adjacent to the afferent fibres at the muscle.

26. The system of claim 22, wherein the probe measurement electrodes are implanted adjacent to the efferent fibres on the ventral side of the spinal cord.

27. The system of claim 22, wherein the probe measurement electrodes are implanted adjacent to the efferent fibres at a ventral root of the spinal cord.

28. The system of claim 22, wherein the probe measurement electrodes are located adjacent to the efferent fibres at the muscle.

29. The system of claim 26, wherein additional probe measurement electrodes are implanted adjacent to the afferent fibres at a dorsal root of the spinal cord.

30. The system of claim 22, wherein the neural responses correspond to H-waves evoked in the one or more efferent fibres.

31. The system of claim 30, wherein the ER value is determined from a maximal amplitude of an evoked H-wave in the response growth curve.

32. The system of claim 22, wherein the RTL is generated by mapping the determined ER value to a corresponding target ECAP value.

33. The system of claim 22, wherein the processor is further configured to program the stimulator device with the generated RTL.

34. The system of claim 33, wherein the stimulator device is further configured to:
apply, via the pulse generator, a therapeutic stimulus to the spinal cord to stimulate one or more afferent fibres associated with the muscle via one or more therapeutic stimulus electrodes of the electrode array as a treatment to relieve the spasticity of the muscle;
measure, via one or more therapeutic measurement electrodes of the electrode array, an intensity of a therapeutic neural response evoked by the therapeutic stimulus in the one or more afferent fibres; and
adjust an intensity of a subsequent therapeutic stimulus based on the measured therapeutic neural response intensity,
wherein the adjustment is based on a feedback signal representing a difference between the measured therapeutic neural response intensity and the determined RTL.

35. The system of claim 34, wherein the stimulator device is further configured to adjust the RTL before applying the subsequent therapeutic stimulus.

36-52. (canceled)
